(19) United States
(12) Patent Application Publication
(10) Pub. No.: US 2024/0148765 A1
(43) Pub. Date: May 9, 2024

(54) COMPOSITIONS AND METHODS FOR TREATING AND/OR PREVENTING THERAPY-RELATED CARDIOMYOPATHY ASSOCIATED WITH NEUTROPHIL INFILTRATION

(71) Applicant: University of Virginia Patent Foundation, Charlottesville, VA (US)

(72) Inventors: Kenneth Walsh, Charlottesville, VA (US); Soichi Sano, Hikone City (JP)

(73) Assignee: University of Virginia Patent Foundation, Charlottesville, VA (US)

(21) Appl. No.: 18/281,528

(22) PCT Filed: Mar. 14, 2022
(86) PCT No.: PCT/US2022/020245

§ 371 Date: Sep. 11, 2023

Related U.S. Application Data
(60) Provisional application No. 63/160,502, filed on Mar. 12, 2021.

(51) Int. Cl. A61K 31/704; A61K 31/282; A61P 9/04

(52) U.S. Cl. CPC A61K 31/704; A61K 31/282; A61P 9/04

(57) ABSTRACT

Provided are methods for treating and/or preventing genotoxic stress-induced cardiac toxicity, which in some embodiments include administering to a subject an effective amount of an inhibitor of t-CH, of neutrophil activation, of neutrophil migration, or any combination thereof. In some embodiments, the genotoxic stress-induced cardiac toxicity results from exposure to one or more anti-tumor and/or anti-cancer therapies, including but not limited to treatment with one or more chemotherapeutics (e.g., doxorubicin) and/or treatment with radiation. In some embodiments, the genotoxic stress-induced cardiac toxicity, the chemotherapy-induced cardiac toxicity, the chemotherapy-induced heart damage, and/or the chemotherapy-induced reduction in cardiac function is characterized by a reduction in cardiac contractility, a thinning of a ventricular wall, a reduction in cardiomyocyte size, or any combination thereof. Also provided are methods for predicting heart failure in subjects previously exposed to a genotoxic agent such as but not limited to a chemotherapeutic by detecting the presence of t-CH in the subject.

### CROSS REFERENCE TO RELATED APPLICATION

The presently disclosed subject matter claims the benefit of U.S. Provisional Patent Application Ser. No. 63/160,502, filed Mar. 12, 2021, the disclosure of which incorporated herein by reference in its entirety.

### GOVERNMENT INTEREST

This invention was made with government support under Grant Nos. HL139819, HL141256, HL138014, and HL152174 awarded by the National Institutes of Health. The government has certain rights in the invention.

### REFERENCE TO SEQUENCE LISTING SUBMITTED ELECTRONICALLY

The content of the electronically submitted sequence listing in ASCII text file (Name: 3062_145_PCT_ST25.txt; Size: 3 kilobytes; and Date of Creation: Mar. 14, 2022) filed with the instant application is incorporated herein by reference in its entirety.

### TECHNICAL FIELD

The presently disclosed subject matter relates to compositions and methods for treating cardiomyopathy, which in some embodiments is cardiomyopathy that results from undesirable neutrophil infiltration into the heart.

### BACKGROUND

Anthracyclines, such as doxorubicin (Dox), are an essential component of many treatment regimens for both solid and hematologic cancers. However, the clinical utility of these drugs is limited by their cardiotoxicity. It is estimated that as many as 10% of patients treated with anthracyclines will develop some form of cardiotoxicity (Zamorano et al., 2016; Campia et al., 2019; Zamorano et al., 2020). Long-term anthracycline cardiotoxicity can occur within 1 year of therapy or can develop years after therapy completion (Yeh & Bickford, 2009), with more recent data suggesting that the early- and late-onset disease may represent different stages of the same evolving phenomenon (Cardinale et al., 2015). Regardless, mortality due to cardiovascular disease (CVD) in cancer survivors is typically greater than that due to cancer itself after a 10-year follow-up (Stoltzfus et al., 2020), and premature CVD is the leading cause of death among aging people who were treated for cancer as children (Fidler et al., 2016; Faber et al., 2018). This chronic cardiotoxicity is considered to be irreversible, refractory to standard heart failure therapy, and associated with very poor prognosis. Thus, there is an obvious need to understand this time-dependent cardiotoxicity and to overcome these hurdles.

Clonal hematopoiesis in the absence of overt hematological abnormalities is caused by precancerous clonal expansions in hematopoietic stem and progenitor cells (HSPCs). These clonal expansions can result from somatic mutations in specific driver genes that confer fitness advantage to the cell (Genovese et al., 2014; Jaiswal et al., 2014). These mutations can be expressed in the progeny leukocytes, contributing to inflammatory processes that appear to promote mortality and morbidity. Age-related clonal hematopoiesis (ARCH) is prevalent in elderly individuals, and it is characterized by the mutations in the genes TET2, DNMT3A, and others, and is associated with an increased risk of cardiovascular disease (CVD) and poor CVD prognosis (Jaiswal et al., 2017; Dorsheimer et al., 2019; Mas-Peiro et al., 2020). We and others have provided experimental evidence that various forms of ARCH can modulate inflammatory responses and contribute to CVD pathology in a gene-specific manner (Jaiswal et al., 2017; Fuster et al., 2017; Sano et al., 2018a; Sano et al., 2018b; Wolach et al., 2018).

In contrast to ARCH, therapy-related CH (t-CH) is an aggressive form of this condition that occurs in individuals who have undergone oncological therapies (Coombs et al., 2018; Gibson et al., 2018). t-CH is typically associated with genes that participate in the DNA damage response (DDR), such as TP53 and PPM1D, that confer resistance to genotoxic stress. Studies indicate that TP53-mutant HSPC pre-exist as small clones that undergo rapid clonal expansion in response to the cytotoxic therapy, likely due to the survival advantage against genotoxic stress that the mutation confers upon the stem cell (Wong et al., 2015; Hsu et al., 2018; Kahn et al., 2018; Bolton et al., 2019). However, whether t-CH is associated with an increased risk of chemotherapy-induced CVD is unknown.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter relates in some embodiments to methods for treating and/or preventing genotoxic stress-induced cardiac toxicity. In some embodiments, the methods comprise, consist essentially of, or consist of administering to a subject in need thereof an effective amount of an inhibitor of therapy-related clonal hematopoiesis (t-CH), neutrophil activation, neutrophil migration, neutrophil extracellular trap formation, neutrophil cytokine and/or chemokine production, or any combination thereof. In some embodiments, the genotoxic stress-induced cardiac toxicity results from exposure to one or more anti-tumor and/or anti-cancer therapies, including but not limited to treatment with radiation and/or treatment with one or more chemotherapeutic agents and/or one or more immunomodulatory agents. In some embodiments, the genotoxic stress-induced cardiac toxicity comprises, consists essentially of, or consists of chemotherapy-induced cardiac toxicity, chemotherapy-induced heart damage, and/or a chemotherapy-induced reduction in cardiac function. In some embodiments, the subject in need thereof has or is at risk for developing genotoxic stress-induced cardiac toxicity, a chemotherapy-induced cardiac toxicity, chemotherapy-induced heart damage, and/or a chemotherapy-induced reduction in cardiac function as a result of treatment with an anthracycline, a platinum compound, a topoisomerase inhibitor, or any combination thereof. In some embodiments, the anthracycline is aclarubicin, daunorubicin, epirubicin, mitoxantrone, valrubicin, or doxorubicin, optionally doxorubicin. In some embodiments, the platinum compound is selected from the group consisting of cisplatin, carboplatin, oxaliplatin, nedaplatin, lobaplatin, triplatin tetranitrate, phenanthriplatin, picoplatin, and satraplatin, or any combination thereof. In some embodiments, the topoisomerase inhibitor is selected from the group consisting of teniposide, irinotecan, etoposide, topotecan, mitoxantrone, moxifloxacin, grepafloxacin, dexrazoxane, valrubicin, and epirubicin, or any combination thereof. In some embodiments, the genotoxic stress-induced cardiac toxicity, the chemotherapy-induced cardiac toxicity, the chemotherapy-induced heart damage, and/or the chemotherapy-induced reduction in cardiac function is characterized by a reduction in cardiac contractility, a thinning of a ventricular wall, a reduction in cardiomyocyte size, or any combination thereof. In some embodiments, the genotoxic stress-induced cardiac toxicity, the chemotherapy-induced cardiac toxicity, the chemotherapy-induced heart damage, and/or the chemotherapy-induced reduction in cardiac function is associated with an inflammatory response, optionally wherein the inflammatory response comprises an induction of one or more of interleukin-10 (IL-10), interleukin-6 (IL-6), and tumor necrosis factor (Tnf). In some embodiments, the subject is a cancer survivor who is relatively young and/or does not have other known cardiovascular risk factors. In some embodiments, the genotoxic stress-induced cardiac toxicity results from exposure to ionizing radiation. In some embodiments, the genotoxic stress-induced cardiac toxicity results from exposure to a platinum compound, a topoisomerase inhibitor, or any combination thereof. In some embodiments, the inhibitor of therapy-related clonal hematopoiesis (t-CH), neutrophil activation, neutrophil migration, neutrophil extracellular trap formation, neutrophil cytokine and/or chemokine production is selected from the group consisting of avacopan, danirixin, AZD5069, nemiralisib, lonodelestat, alvelestat, and elafin.

The presently disclosed subject matter also relates in some embodiments to methods for predicting heart failure in subject previously treated with a genotoxic-stress-inducing agent such as but not limited to a chemotherapeutic. In some embodiments, the methods comprise, consist essentially of, or consist of detecting the presence of therapy-related clonal hematopoiesis (t-CH) in the subject, wherein the presence of t-CH in the subject is predictive of heart failure in the subject. In some embodiments, the genotoxic-stress-inducing agent is a chemotherapeutic such as an anthracycline. In some embodiments, the anthracycline is aclarubicin, daunorubicin, epirubicin, mitoxantrone, valrubicin, or doxorubicin, optionally doxorubicin. In some embodiments, the platinum compound is selected from the group consisting of cisplatin, carboplatin, oxaliplatin, nedaplatin, lobaplatin, triplatin tetranitrate, phenanthriplatin, picoplatin, and satraplatin, or any combination thereof. In some embodiments, the topoisomerase inhibitor is selected from the group consisting of teniposide, irinotecan, etoposide, topotecan, mitoxantrone, moxifloxacin, grepafloxacin, dexrazoxane, valrubicin, and epirubicin, or any combination thereof. In some embodiments, the genotoxic agent comprises, consists essentially of, or consists of ionizing radiation.

In some embodiments of the presently disclosed methods, the subject is a mammal, optionally a human. In some embodiments, the subject is a cancer survivor who is relatively young and/or does not have other known cardiovascular risk factors.

In some embodiments, the presently disclosed subject matter also relates to uses of compositions comprising, consisting essentially of, or consisting of one or more inhibitors of therapy-related clonal hematopoiesis (t-CH), neutrophil activation, neutrophil migration, neutrophil extracellular trap formation, neutrophil cytokine and/or chemokine production, or any combination thereof for treating or preventing genotoxic stress-induced cardiac toxicity, chemotherapy-induced cardiac toxicity, chemotherapy-induced heart damage, and/or chemotherapy-induced reduction in cardiac function

Accordingly, it is an object of the presently disclosed subject matter to provide compositions and methods for treating and/or preventing genotoxic-stress-induced cardiac toxicity, heart damage, or a reduction in cardiac function, such as but not limited to cardiac toxicity, heart damage, and/or a reduction in cardiac function resulting from chemotherapy.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following Description, Figures, and Examples.

### BRIEF DESCRIPTION OF THE FIGURES

FIGS. 1A-1G. Dox treatment promotes the expansion of Trp53 mutant blood hematopoietic cells subsets, but this cell expansion is not associated with changes in absolute blood cell number. However, Dox-mediated acceleration of Trp53 mutant clones promotes doxorubicin-related cardiac toxicity. FIG. 1A. Schematic of adoptive bone marrow transplantation and Dox administration. A total of 1.5×10⁷ unfractionated donor bone marrow cells were sequentially injected to non-preconditioned B6.SJL-CD45.1 recipients over 3 consecutive days (indicated by small arrowheads). Donor cells were obtained from either C57BL/6J wild type mice (Trp53^{+/-}) or Trp53-heterozygous mice (Trp53^{+/-}). Recipient mice were injected with Dox at 7 weeks after BMT involving 4 rounds of 6 μg/g i.p. injection (2 μg/g/day over 3 consecutive days), with 3 weeks between each round (indicated by large arrow heads). Absolute number and the chimerism of test cells in peripheral blood at baseline and after each cycle of Dox or saline administration were evaluated by sequential flow cytometry analysis. FIG. 1B. Flow cytometry analysis of blood chimerism over the time course to show the progressive expansion of Trp53^{+/-} mutant clones in total WBC, neutrophil and Ly6Chi monocytes after Dox treatment compared to saline administration (n=6-7 per group). The approximate times of multiple Dox injections are indicated. Statistical analysis was performed with two-way ANOVA with Tukey multiple-comparison tests. FIG. 1C. Flow cytometry analysis of blood chimerism over a time course to show the progressive expansion of Trp53^{+/-} mutant clones compared to wild-type in ly6C^{lo} monocytes, B cells, CD4⁺ T cells and CD8⁺ T cells after 4 rounds of Dox treatment (total 24 μg/g) compared to saline administration (indicated by arrows) in the adoptive transplantation model. FIG. 1D. Dox exposure promotes the expansion of Trp53 mutant hematopoietic stem and progenitor cells. Flow cytometry analysis of bone marrow at 19 weeks after adoptive BMT showing increased chimerism (%) of donor-derived LT-HSC, LSK and GMP in mice transplanted with Trp53-heterozygous deficient cells compared to wild-type after 4 rounds of Dox or saline treatment (n=6-7 per group). LT-HSC=long-term hematopoietic stem cell; GMP=granulocyte-monocyte progenitor. FIG. 1E. Trp53-mediated cell expansion is not associated with changes in absolute blood cell number. Analysis of peripheral blood cell numbers in mice from different experimental groups: wild-type (WT)+Saline in black, WT+Dox in blue, Trp53^{+/-} Saline in yellow, Trp53^{+/-} Dox in red. Hemoglobin and platelet levels were also assessed in the same experimental groups. Statistical analysis was performed with two-way ANOVA with Tukey multiple-comparison tests. (n=6-8 per group). Statistical analysis was performed with 2-way ANOVA with Tukey multiple-comparison tests. (n=6-7 per group) *p<0.05, ***p<0.001, ****p<0.0001. FIG. 1F. Echocardiographic analyses of fractional shortening (FS, %) and left ventricular posterior wall thickness diameter (LVPWTd, mm) of mice transplanted with wild-type cells or Trp53-insufficient cells at baseline and after four cycles of Dox or saline administration (n=9-12 per group). Statistical analysis was performed with two-way ANOVA with Tukey multiple-comparison tests. FIG. 1G. Heart weight (HW) was adjusted by tibia length (TL) (n=9-12 per group) at the end of study. Statistical analysis was performed with two-way ANOVA with Tukey multiple-comparison tests. **p<0.01, ***p<0.001, ****p<0.0001.

FIGS. 2A-2C. Dox exposure promotes the expansion of Trp53^{R270H} mutant cells into hematopoietic cell subsets in the adoptive bone marrow transplantation model, leading to accelerated Dox toxicity of the heart. FIG. 2A. Flow cytometry analysis of blood chimerism over the time reveals the progressive expansion of Trp53^{R27OH} mutant clones in total WBC, neutrophil, Ly6C^{hi} monocyte, Ly6C^{lo} monocytes, B cells, CD4⁺ T cells and CD8⁺ T cells after 4 rounds of Dox compared to saline treatment (2 μg/g/day over 3 consecutive days per round, with 3 weeks' interval between each round). Timing of Dox/saline administration is indicated by arrows. FIG. 2B. Dox exposure promotes the expansion of Trp53^{R27OH} mutant hematopoietic stem and progenitor cells. Flow cytometry analysis of bone marrow at 20 weeks after adoptive BMT showing increased chimerism (%) of donor-derived LSK and GMP, and an increase trend of LT-HSC in mice transplanted with Trp53^{R27OH} vs. wild-type cells after Dox. Saline treatment had no effect on cell expansion in the Trp53^{R27OH} vs. wild-type group. LT-HSC=long-term hematopoietic stem cell; GMP=granulocyte-monocyte progenitor. FIG. 2C. The expansion of Trp53^{R27OH} cells in blood accelerates Dox cardiotoxicity in mice. Echocardiographic analyses of fractional shortening (FS, %) and left ventricular posterior wall thickness diameter (LVPWTd, mm) of mice transplanted with wild-type cells or Trp53^{R27OH}-mutated cells at baseline and after four cycles of Dox or saline administration. Measurements were made at the 20 week time point after adoptive BMT. Statistical analysis was performed with two-way ANOVA with Tukey multiple-comparison tests. (n=10-11 per group). *p<0.05, **p<0.01, ***p<0.001, ****p<0.0001.

FIGS. 3A-3C. Trp53-mutated hematopoietic cells accelerate Dox cardiotoxicity and enhance myocardial inflammation in conditioned mice. FIG. 3A. Analyses of fractional shortening (FS, %) and left ventricular posterior wall thickness diameter (LVPWTd, mm) of lethally irradiated mice transplanted with 30% wild-type cells or 30% Trp53-heterozygous cells. After 4 weeks of recovery mice were treated and with Dox or saline for 8 weeks. Dox or saline control was administered intraperitoneally by a sterile syringe and needle (29G) with a dose of 15 μg/g (body weight) split into 3 injections over 10 days. Statistical analysis was performed with two-way repeated-measures ANOVA with Sidak multiple-comparison tests. Echocardiography was performed at indicated time points post-BMT. FIG. 3B. Quantification analysis of cross-sectional area (CSA) of cardiomyocyte by wheat germ agglutinin staining of the heart sections from each experimental group (Trp53^{+/+} in White, Trp53+ in Blue) at the end of the study. Statistical analysis was performed with unpaired (two-tailed) Student t test. FIG. 3C. Real time qPCR analysis of transcript expression in hearts from the different experimental groups. 36b4 was used as a reference control. Statistical analysis was performed with unpaired (two-tailed) Student t test with Welch correction (Il-1b) and Mann-Whitney U test (11-6, Tnf). For a-c, n=10-11 per genotype. *p<0.05, **p<0.01, ****p<0.0001.

FIGS. 4A-4E. Dynamic changes in cardiac immune cells following Dox administration to wild-type mice. FIG. 4A. Schematic of the study. C57BL6/J mice were administered a single injection of Dox (15 μg/g) intraperitoneally at indicated time points. Mice in non-treated (NT) group were injected with sterile saline. FIG. 4B. Body weight (BW, g) and heart weight (HW, mg) of mice from each group at the end of study (n=5 per time point). And echocardiographic analysis of left ventricular posterior wall thickness diameter (LVPWTd, mm) and ejection fraction (EF, %) of mice at baseline and 14 days post-Dox or saline administration (n=4). Statistical analysis was performed with Kruskal-Wallis H test with Dunn's multiple-comparison tests for BW and HW; and two-way repeated-measures ANOVA with Sidak multiple-comparison tests for LVPWTd and EF. FIG. 4C. Analysis of peripheral blood neutrophil (Neut), monocytes (Mono) and lymphocyte (Lymph) levels in mice from each experimental group (n=5 per group). Statistical analysis was performed with one-way ANOVA with Tukey multiple-comparison tests. FIG. 4D. Flow cytometry analysis of cardiac immune cells from C57BL6/J wild type mice after Dox treatment (single injection of 15 μg/g) at different time points (0, 3, 7, 14 days post injection respectively, n=5 per group). NT is “non-treated”. Statistical analysis was performed with one-way ANOVA with Tukey multiple-comparison tests. FIG. 4E. Flow cytometry analysis of cardiac immune cells from mice that underwent competitive BMT and Dox administration. In this study, mice underwent partial (30%) bone marrow reconstitution with Trp53 deficient cells (30%-Het BMT mice) or WT cells (30%-WT BMT mice). After 7 weeks' recovery from BMT, mice were injected intraperitoneally with Dox (single injection of 15 μg/g) and the hearts were harvested 7 days later for flow cytometry analysis of resident immune cells (n=6). Statistical analysis was performed with un-paired (two-tailed) Student's t test (Neutrophil) and Mann-Whitney U tests (Ly6C^{hi} monocyte, macrophage). *p<0.05, **p<0.01, ***p<0.001, ****p<0.0001. NS=not significant.

FIGS. 5A-5C. Trp53-deficient neutrophils display a distinct gene expression profile. FIG. 5A. Principal component analysis (PCA) of ultra-low input RNA sequencing data obtained from sorted donor bone marrow-derived, peripheral blood neutrophils of Trp53-sufficient mice and Trp53-heterozygous deficient mice at 1 day after Dox injection (n=4 per genotype). FIG. 5B. Volcano plots showing the number of genes differently expressed between peripheral blood neutrophil from Trp53-sufficient vs. Trp53-heterozygous deficient mice at 1 day after Dox injection. FIG. 5C. Heat map of select group of the most highly differentially expressed genes from the highest ranked annotation categories comparing blood neutrophils between Trp53-sufficient (Trp53^{+/+}) and Trp53-heterozygous deficient mice (Trp53^{+/-}) at 1 day after Dox administration (15 μg/g single injection).

FIGS. 6A-6E. Negligible contribution of monocytes to Dox-induced cardiotoxicity in wild type mice, as neutrophil depletion inhibits Dox-induced cardiotoxicity in wild type mice and a neutrophil recruitment inhibitor diminishes Dox-induced cardiotoxicity in wild-type mice. FIG. 6A. Echocardiographic analyses of fractional shortening (FS, %) and left ventricular posterior wall thickness diameter (LVPWTd, mm) of wild-type (CCR2^{+/+}) and CCR2-knockout (CCR2^{GFP/GFP}) mice at baseline and 4 weeks after Dox injection with a dose of 15 μg/g (body weight) split into 3 injections over 10 days (n=7-9 in each group). Statistical analysis was performed with two-way repeated-measures ANOVA with Sidak multiple-comparison tests. FIG. 6B. Echocardiographic analysis of left ventricular posterior wall thickness diameter (LVPWTd, mm) and fractional shortening (FS, %) of C57BL6/J mice at 2 weeks after treatment with sterile saline, or Dox+isotype control, or Dox+anti-Ly6G. 15 μg/g Dox (split into 3 injections) was intraperitoneally administered over a period of 10 days. The antibody was administered via intraperitoneal injection every 3 days (500 μg/injection/mouse) starting 2 days prior to Dox administration and continued until end of the study. Statistical analysis was performed with one-way ANOVA with Tukey multiple-comparison tests (LVPWTd) and Kruskal-Wallis H test with Dunn's multiple-comparison tests (FS). FIG. 6C. Real time qPCR analysis of transcript expression in heart tissues of mice from each experimental group. 36b4 was used as a reference control. FIG. 6D. Relative immunofluorescence values of 3-nitrotyrosine staining in heart sections from the different experimental groups. For FIGS. 6B-6D, n=4-7 in each group. Statistical analysis was performed with Kruskal-Wallis H test with Dunn's multiple-comparison tests. *p<0.05, **p<0.01, ***p<0.001. FIG. 6E. In this experiment, C57BL/6J mice received Dox via an intraperitoneal injection (15 μg/g split into 3 injections over 10 days) in the presence or absence of a CXCR2 inhibitor. The CXCR2 inhibitor was administered at a dose of 2 mg/kg/day via intraperitoneal injection initiating 1 day prior to Dox administration and continuing until the end of study. Control mice received an equivalent volume of the DMSO solvent for the SB265610. Echocardiographic analyses of left ventricular posterior wall thickness diameter (LVPWTd, mm) and fractional shortening (FS, %) of mice was determined at the 14-day timepoint. n=6-7 per group. Statistical analysis was performed with two-way repeated-measures ANOVA with Sidak multiple-comparison tests. ***p<0.001.

FIGS. 7A-7C. Trp53-defficient myeloid cells accelerates Dox-induced cardiotoxicity. Mice were administered Dox by intraperitoneal injection (15 μg/g split into 3 injections over 10 days). FIG. 7A. Echocardiographic analysis of fractional shortening (FS, %) of myeloid-specific Trp53-defficient mice (Lyz2^{Cre/+}Tp53_{fl/f}) and littermate controls (Control) before and at 8 weeks after Dox administration. Statistical analysis was performed with two-way repeated-measures ANOVA with Sidak multiple-comparison tests. FIG. 7B. Survival of myeloid-specific Trp53-defficient mice (Lyz2^{Cre/+}Tp53^{fl/f}) and littermate controls (Control) after Dox administration. Survival curve was obtained by the Kaplan-Meier method. Statistical analysis was performed with log-rank test. FIG. 7C. Real-time qPCR analysis of transcript expression in the myocardium obtained from Lyz2^{Cre/+}Trp53^{fl/fl} and littermate control mice at 8 weeks after Dox treatment (n=7 per genotype). Statistical analysis was performed with unpaired (two-tailed) Student t test (11-6, Il-1b, b/a MHC), or unpaired (two-tailed) Student t test with Welch correction (Nppa), or Mann-Whitney U test (Tnf).

FIGS. 8A-8D. Neutrophil overactivation contributes to the cardiac toxicity induced by Trp53-mediated clonal hematopoiesis. FIG. 8A. Schematic of the study in FIGS. 8B-8D. Lethally irradiated mice underwent a competitive BMT with 30% Trp53-heterozygous cells (Trp53^{+/-}). After a 6 week recovery from BMT, mice received an intraperitoneal administration of anti-Ly6G (clone 1A8, neutrophil depletion) or isotype control (clone 2A3) antibody (Ab: 500 μg/injection/mouse, 1 injection/3 days for 4 weeks) from 1 day before Dox administration (15 μg/g, split into 3 injections over 10 days) until the end of this study. FIG. 8B. Heart weight (HW) adjusted to tibia length (TL), FIG. 8C. left ventricular posterior wall thickness diameter (LVPWTd, mm; left panel) and fractional shortening (FS, %; right panel), and FIG. 8D. Relative immunofluorescence values of 3-nitrotyrosine staining in heart sections of mice. All mice were treated with sterile saline, or Dox+isotype control, or Dox+anti-Ly6G. Statistical analysis was performed with two-way ANOVA with Tukey multiple-comparison tests. For e-g, n=5-8 per group. *p<0.05, **p<0.01, ***p<0.001, ****p<0.0001. NS=not significant.

FIG. 9 is a schematic diagram of a mechanism by which hematopoietic stem and progenitor cells (HSPCs) present in the bone marrow can undergo genotoxic stress including but not limited to genotoxic stress resulting from exposure to chemotherapeutic drugs (e.g., doxorubicin), which can result in the production of mutated HSPCs. Mutated HSPCs can then undergo clonal selection, ultimately leading to the production of mutated blood cells including mutated neutrophils. These mutated, clonally selected neutrophils can, in response to inflammation and reactive oxygen species (ROS) production traffic to and/or other wise accumulate in the cardiac muscle, which can lead to increased cardiotoxicity.

### DETAILED DESCRIPTION

### I. General Considerations

Therapy-related clonal hematopoiesis (t-CH) is often observed in cancer survivors. This form of clonal hematopoiesis typically involves somatic mutations in driver genes that encode components of the DNA damage response (DDR) and confer hematopoietic stem and progenitor cells (HSPC) with resistance to the genotoxic stress of the cancer therapy. A model of t-CH was established through the transfer of Trp53-mutant HSPC to mice followed by treatment with a course of the chemotherapeutic agent doxorubicin. Comparing wild-type and Trp53-mutant HSPC, these studies revealed that neutrophil infiltration into the heart significantly contributes to doxorubicin-induced cardiac toxicity and that this condition is amplified in the model of Trp53-mediated t-CH. These data suggest that (1) t-CH could contribute to the elevated heart failure risk that occurs in cancer survivors; and (2) neutrophil infiltration into the heart by whatever mechanism or etiology could contribute to cardiac toxicity generally.

### II. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

A disease or disorder is “alleviated” if the severity of a symptom of the disease, condition, or disorder, or the frequency with which such a symptom is experienced by a subject, or both, are reduced.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As use herein, the terms “administration of” and or “administering” a compound should be understood to mean providing a compound of the presently disclosed subject matter or a prodrug of a compound of the presently disclosed subject matter to a subject in need of treatment.

As used herein, the term “aerosol” refers to suspension in the air. In particular, aerosol refers to the particlization or atomization of a formulation of the presently disclosed subject matter and its suspension in the air.

As used herein, an “analog” of a chemical compound is a compound that, by way of example, resembles another in structure but is not necessarily an isomer (e.g., 5-fluorouracil is an analog of thymine).

As used herein, “amino acids” are represented by the full name thereof, by the three letter code corresponding thereto, or by the one-letter code corresponding thereto, as indicated in Table 1.

TABLE 1
Amino Acids, Codes, and Functionally Equivalent Codons

| | 3-Letter | 1-Letter | |
|---|---|---|---|
| Full Name | Code | Code | Functionally Equivalent Codons |
| Aspartic Acid | Asp | D | GAC GAU |
| Glutamic Acid | Glu | E | GAA GAG |
| Lysine | Lys | K | AAA AAG |
| Arginine | Arg | R | AGA AGG CGA CGC CGG CGU |
| Histidine | His | H | CAC CAU |
| Tyrosine | Tyr | Y | UAC UAU |
| Cysteine | Cys | C | UGC UGU |
| Asparagine | Asn | N | AAC AAU |
| Glutamine | Gln | Q | CAA CAG |
| Serine | Ser | S | ACG AGU UCA UCC UCG UCU |
| Threonine | Thr | T | ACA ACC ACG ACU |
| Glycine | Gly | G | GGA GGC GGG GGU |
| Alanine | Ala | A | GCA GCC GCG GCU |
| Valine | Val | V | GUA GUC GUG GUU |
| Leucine | Leu | L | UUA UUG CUA CUC CUG CUU |
| Isoleucine | Ile | I | AUA AUC AUU |
| Methionine | Met | M | AUG |
| Proline | Pro | P | CCA CCC CCG CCU |
| Phenylalanine | Phe | F | UUC UUU |
| Tryptophan | Trp | W | UGG |

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as exchanges within one of the following five groups:
I. Small aliphatic, nonpolar, or slightly polar residues: Ala, Ser, Thr, Pro, Gly;
II. Polar, negatively charged residues and their amides: Asp, Asn, Glu, Gln;
III. Polar, positively charged residues: His, Arg, Lys;
IV. Large, aliphatic, nonpolar residues: Met Leu, Ile, Val, Cys
V. Large, aromatic residues: Phe, Tyr, Trp

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino-and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody”, as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms including, for example, polyclonal antibodies, monoclonal antibodies, Fv, Fab and F(ab)2, as well as single chain antibodies and humanized antibodies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates. The term “immunogen” is used interchangeably with “antigen” herein.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this presently disclosed subject matter, and is effective in killing or substantially inhibiting the growth of microbes. “Antimicrobial” as used herein, includes antibacterial, antifungal, and antiviral agents.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the peptides encompasses natural or synthetic portions of a longer peptide or protein that are capable of specific binding to their natural ligand or of performing the desired function of the protein, for example, a fragment of a protein of larger peptide which still contains the epitope of interest and is immunogenic.

The term “biological sample”, as used herein, refers to samples obtained from a subject, including, but not limited to, skin, hair, tissue, blood, plasma, cells, sweat and urine.

As used herein, the term “carrier molecule” refers to any molecule that is chemically conjugated to the antigen of interest that enables an immune response resulting in antibodies specific to the native antigen.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and in some embodiments at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

As used herein, a “derivative” of a bacterium, antigen, composition or other compound refers to a bacterium, antigen, composition or other compound that may be produced from bacterium, antigen, composition or other compound of similar structure in one or more steps.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting a risk or propensity to an addictive related disease disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular, and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly at least five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95%, and in some embodiments at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be in some embodiments at least about 20 nucleotides in length, in some embodiments at least about 50 nucleotides, in some embodiments from about 50 to about 100 nucleotides, in some embodiments at least about 100 to about 200 nucleotides, in some embodiments at least about 200 nucleotides to about 300 nucleotides, in some embodiments at least about 300 to about 350, in some embodiments at least about 350 nucleotides to about 500 nucleotides, in some embodiments at least about 500 to about 600, in some embodiments at least about 600 nucleotides to about 620 nucleotides, in some embodiments at least about 620 to about 650, and in some embodiments the nucleic acid fragment will be greater than about 650 nucleotides in length.

The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993. This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

By the term “immunizing a subject against an antigen” is meant administering to the subject a composition, a protein complex, a DNA encoding a protein complex, an antibody or a DNA encoding an antibody, which elicits an immune response in the subject, and, for example, provides protection to the subject against a disease caused by the antigen or which prevents the function of the antigen.

The term “immunologically active fragments thereof” will generally be understood in the art to refer to a fragment of a polypeptide antigen comprising at least an epitope, which means that the fragment at least comprises 4 contiguous amino acids from the sequence of the polypeptide antigen.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit”, as used herein when referring to a function, refers to the ability of a compound of the presently disclosed subject matter to reduce or impede a described function. In some embodiments, inhibition is by at least 10%, in some embodiments by at least 25%, in some embodiments by at least 50%, and in some embodiments, the function is inhibited by at least 75%. When the term “inhibit” is used more generally, such as “inhibit Factor I”, it refers to inhibiting expression, levels, and activity of Factor I.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting, or applying, or administering” includes administration of a compound of the presently disclosed subject matter by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, or rectal approaches.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to, through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

The term “nasal administration” in all its grammatical forms refers to administration of at least one compound of the presently disclosed subject matter through the nasal mucous membrane to the bloodstream for systemic delivery of at least one compound of the presently disclosed subject matter. The advantages of nasal administration for delivery are that it does not require injection using a syringe and needle, it avoids necrosis that can accompany intramuscular administration of drugs, trans-mucosal administration of a drug is highly amenable to self administration, and intranasal administration of antigens exposes the antigen to a mucosal compartment rich in surrounding lymphoid tissues, which can promote the development of a more potent immune response, particularly more potent mucosal immune responses.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides but when used in the context of a longer amino acid sequence can also refer to a longer polypeptide.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder. “Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of contracting the disease and/or developing a pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-p-galactoside to the medium (Gerhardt et al., 1994).

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds to”, as used herein, is meant when a compound or ligand functions in a binding reaction or assay conditions which is determinative of the presence of the compound in a sample of heterogeneous compounds.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include in some embodiments mammals, which in some embodiments can be a human.

As used herein, a “subject in need thereof” is a patient, animal, mammal, or human, who will benefit from the method of this presently disclosed subject matter.

As used herein, “substantially homologous amino acid sequences” includes those amino acid sequences which have in some embodiments at least about 95% homology, in some embodiments at least about 96% homology, in some embodiments at least about 97% homology, in some embodiments at least about 98% homology, and in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: in some embodiments 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when it is in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat”, as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### III. Methods and Compositions

In some embodiments, the presently disclosed subject matter relates to methods for treating and/or preventing genotoxic stress-induced cardiac toxicity. In some embodiments, the methods comprise, consist essentially of, or consist of administering to a subject in need thereof an effective amount of an inhibitor of therapy-related clonal hematopoiesis (t-CH), neutrophil activation, neutrophil migration, neutrophil extracellular trap formation (referred to as NETosis), neutrophil cytokine and/or chemokine production, or any combination thereof.

As used herein, the phrase “genotoxic stress-induced cardiac toxicity” refers to any disease, disorder, or condition at least one symptom or consequence of which is associated with exposure of a subject to genotoxic stress. As used herein, the phrase “genotoxic stress” refers to genotoxin-induced cellular DNA damage, which is a major cause of human and veterinary illness. Illnesses attributable to genotoxic stress include aging, cancer, and some forms of heart failure. Genotoxins that induce such cellular DNA damage include ultraviolet light, ionizing radiation, and chemotherapy agents including anthracyclines, platinum compounds, topoisomerase inhibitors, including but type I and type II topoisomerase inhibitors, and cyclophosphamide. Exemplary anthracyclines include aclarubicin, daunorubicin, epirubicin, mitoxantrone, valrubicin, and doxorubicin; exemplary platinum compounds include cisplatin, carboplatin, oxaliplatin, nedaplatin, lobaplatin, triplatin tetranitrate, phenanthriplatin, picoplatin, and satraplatin; and exemplary topoisomerase inhibitor is selected from the group consisting of teniposide, irinotecan, etoposide, topotecan, mitoxantrone, moxifloxacin, grepafloxacin, dexrazoxane, valrubicin, and epirubicin.

Any inhibitor of therapy-related clonal hematopoiesis (t-CH), neutrophil activation, neutrophil migration, neutrophil extracellular trap formation, neutrophil cytokine and/or chemokine production, and any combination thereof, can be employed in the compositions and methods of the presently disclosed subject matter. Non-limiting examples of such inhibitors include avacopan ((2R,3S)-2-[4-(cyclopentylamino)phenyl]-1-(2-fluoro-6-methylbenzoyl)-N-[4-methyl-3-(trifluoromethyl)phenyl]piperidine-3-carboxamide), danirixin (1-[4-chloro-2-hydroxy-3-[(3S)-piperidin-3-yl]sulfonylphenyl]-3-(3-fluoro-2-methylphenyl)urea), AZD5069 (N-(2-((2,3-difluorobenzyl)thio)-6-(((2R,3S)-3,4-dihydroxybutan-2-yl)oxy)pyrimidin-4-yl)azetidine-1-sulfonamide), nemiralisib (2-[6-(1H-indol-4-yl)-1H-indazol-4-yl]-5-[(4-propan-2-ylpiperazin-1-yl)methyl]-1,3-oxazole), lonodelestat (cyclo[Ala-Ser-Ile-Pro-Pro-Gln-Lys-Tyr-D-Pro-Pro-Nle(Bu)-Glu-Thr; SEQ ID NO: 15]), alvelestat (6-methyl-5-(2-methylpyrazol-3-yl)-N-[(5-methylsulfonylpyridin-2-yl)methyl]-2-oxo-1-[3-(trifluoromethyl)phenyl]pyridine-3-carboxamide), and elafin ((2S)-2-[[(2S)-1-[(2S)-2-[[(2S)-2-[[(1R,4S,4aS,7S,7aS,10S,10aS,13aR,16S,20aS,22S,23aS,25S,26aS,28S,29aS,31S,32aS,34R,37S,40S,43 S,46S,49S,52S,58R,63R,66S,72S,75R ,81S,84S,87R,92R,98S)-58-[[(2S)-2-[[2-[[(2S)-1-[(2S)-6-amino-2-[[(2S,3R)-2-[[(2S)-2-[[(2S)-2-[[(2S)-1-[2-[[(2S)-6-amino-2-[[(2S)-2-[[(2S)-1-[(2S)-2-[[(2S)-5-amino-2-[[(2S)-2-aminopropanoyl]amino]-5-oxopentanoyl]amino]-4-carboxybutanoyl]pyrrolidine-2-carbonyl]amino]-3-methylbutanoyl]amino]hexanoyl]amino]acetyl]pyrrolidine-2-carbonyl]amino]-3-methylbutanoyl]amino]-3-hydroxypropanoyl]amino]-3-hydroxybutanoyl]amino]hexanoyl]pyrrolidine-2-carbonyl]amino]acetyl]amino]-3-hydroxypropanoyl]amino]-7a,10a,23a-tris(4-aminobutyl)-7,22-bis(2-amino-2-oxoethyl)-4a,40,46,49-tetrakis[(2S)-butan-2-yl]-4,37-bis(3-carbamimidamidopropyl)-66-(2-carboxyethyl)-26a,32a-bis(carboxymethyl)-29a-[(1R)-1-hydroxyethyl]-72-(hydroxymethyl)-31,84-dimethyl-20a,25,43-tris(2-methylpropyl)-28,81-bis(2-methylsulfanylethyl)-2a,3,5a,6,8a,9,11a,15,18a,21,21a,24,24a,27,27a,30,30a,33,33a, 35a,36,39,42,45,48,51,57,64,67,70,73,76,79,82,85,93,99-heptatriacontaoxo-15a,16a,38a,39a,60,61,89,90-octathia-a,2,3a,5,6a,8,9a,12a,14,19a,20,22a,23,25a,26,28a, 29,31a,32,34a,35,36a,38,41,44,47,50,56,65,68,71,74,77,80,83,86,94-heptatriacontazaoctacyclo[90.25.17.434,75.263,113.010,14.016,20.052,56.094,98]tetracon tahectane-87-carbonyl]amino]-3-phenylpropanoyl]amino]-3-methylbutanoyl]pyrrolidine-2-carbonyl]amino]-5-amino-5-oxopentanoic acid). See e.g., Leslie, 2020 for a discussion of compounds with activity in inhibiting various neutrophil biological activities.

Neutrophil activation can have various undesirable consequences, several of which result from proinflammatory signaling (i.e., the release of reactive oxygen intermediates, nitric oxide, proteases, matrix metalloproteinases, cytokines, etc.) and the suppression or delay of neutrophil programmed cell death. Neutrophils are endstage cells and undergo apoptosis upon release into the circulation. However, during inflammatory diseases, neutrophil apoptosis is suppressed (see e.g., Jimenez et al., 1997; Taneja et al., 2004). Enhanced neutrophil survival at the site of inflammation promotes increased bactericidal activity and can also result in acute inflammatory damage. Tumor Necrosis Factor (TNF) and other proinflammatory cytokines are important regulators of neutrophil function during such inflammatory responses through activation of proinflammatory signaling and are involved in the suppression of neutrophil apoptosis (Lee et al., 1993; Kilpatrick et al., 2002).

In some embodiments, genotoxic stress in a subject is a consequence of the subject undergoing some form of therapy for a disease, disorder, and/or condition, such as but not limited to an anti-tumor and/or anti-cancer therapy. Thus, in some embodiments the genotoxic stress results from anti-tumor and/or anti-cancer therapy, which in some embodiments can lead to cardiac toxicity. Exemplary anti-tumor and/or anti-cancer therapies that can induce genotoxic stress include chemotherapeutic treatments, radiation (including but not limited to ionizing radiation), and treatments with other immunomodulatory compositions including but not limited to checkpoint inhibitors and cytokines. By way of example and not limitation, anti-tumor and/or anti-cancer therapies that can induce genotoxic stress include any of the following, either alone or in combination: anti-CA125 (or oregovomab Mab B43.13), anti-idiotype Ab (ACA-125), anti-HER-2 (trastuzumab, pertuzumab), anti-MUC-1 idiotypic Ab (HMFG1), HER-2/neu peptide, NY-ESO-1, anti-Programed Death-1 (“PD1”) (or PD1-antagonists such as BMS-936558), anti-CTLA-4 (or CTLA-4 antagonists), vermurafenib, ipilimumab, dacarbazine, IL-2, IFN-α, IFN-γ, temozolomide, receptor tyrosine kinase inhibitors (e.g., imatinib, gefitinib, erlotinib, sunitinib, tyrphostins, telatinib), sipileucel-T, platinum-based agents (e.g., carboplatin, cisplatin, and/or oxaliplatin), taxanes (e.g., paclitaxel, docetaxel, larotaxel, and/or cabazitaxel), alkylating agents, antimetabolites and/or vinca alkaloids, topoisomerase inhibitors (e.g., teniposide, irinotecan, etoposide, topotecan, mitoxantrone, moxifloxacin, grepafloxacin, dexrazoxane, valrubicin, and epirubicin), or combinations thereof. More particularly, in some embodiments the anti-tumor and/or anti-cancer therapy can include treatment with one or more chemotherapeutic agents, e.g., platinum-based agents, taxanes (e.g., paclitaxel, docetaxel, larotaxel, and/or cabazitaxel), alkylating agents, anthracyclines (e.g., daunorubicin, doxorubicin (e.g., liposomal doxorubicin), epirubicin, valrubicin, and/or idarubicin), antimetabolites (e.g., an antifolate (e.g., pemetrexed, floxuridine, raltitrexed) and a pyrimidine analogue (e.g., capecitabine, cytrarabine, gemcitabine, 5FU)), vinca alkaloids (e.g., vinblastine, vincristine, vindesine, vinorelbine), vascular endothelial growth factor (VEGF) pathway inhibitors, epidermal growth factor (EGF) pathway inhibitors.

In some embodiments, the genotoxic stress results from an anti-tumor and/or anti-cancer therapy that comprises, consists essentially of, or consists of radiation treatment. Exemplary radiations treatments include external radiation (e.g., external beam radiation), internal radiation (e.g., brachytherapy), and systemic radiation. By way of example and not limitation, external beam radiation therapy consists of three main stages. First, a precise three-dimensional map of the anatomical structures in the location of interest (target volume) is constructed using conventional three-dimensional imaging technology, such as computed tomography (CT) or magnetic resonance imaging (MRI). Second, a treatment plan is developed for delivering a predefined dose distribution to the target volume that is acceptable to the clinician. Finally, the treatment plan is executed using an accepted beam delivery apparatus. The basic strategy of external beam radiation therapy is to utilize multiple beams of radiation from multiple directions to “cross-fire” at the target volume. In that way, radiation exposure to normal tissue is kept at relatively low levels, while the dose to the tumor cells is enhanced. As such, an objective of external beam radiation involves designing a beam profile, for example, a collection of beams, that delivers a necrotic dose of radiation to the tumor volume, while the dose delivered to nearby critical structures and surrounding normal tissue is kept below established tolerance levels. However, in some embodiments delivery to nearby critical structures and surrounding normal tissue nonetheless occurs, and those structures and tissues can experience genotoxic stress and/or damage.

Alternatively, in some embodiments the radiation therapy is total body irradiation (TBI). TBI can involve irradiation of the entire body or, in some embodiments, the entire body but with the lungs partially shielded. Total body irradiation can be administered at various doses. For example, total body irradiation can be administered from about 10 to about 12 Gy or more. In some embodiments, total body irradiation can be fractionated, with smaller doses delivered in several sessions, rather than delivering the entire dose at once. In some cases, 1, 2, 3, 4, 5, 6, 7, 8, 9, or up to 10 different doses of irradiation can be administered. Here as well, TBI can induce significant undesired genotoxic stress and/or damage in structures and tissues.

Brachytherapy involves introducing a radiation source directly into a target tissue, which can include a tumor and/or tissue surrounding a cavity or void, which may contain potentially cancerous cells (such as a cavity or void created by removing a tumor). Even though brachytherapy treatments are designed to minimize damage to surrounding structures and tissues, it can occur and thus genotoxic stress remains a concern.

Accordingly, in some embodiments the genotoxic stress-induced cardiac toxicity results from exposure to one or more chemotherapeutics and/or one or more radiation therapies. Non-limiting examples of genotoxic stress-induced cardiac toxicity include chemotherapy-induced cardiac toxicity, chemotherapy-induced heart damage, and/or a chemotherapy-induced reduction in cardiac function. In some embodiments, the subject in need thereof has or is at risk for developing chemotherapy-induced cardiac toxicity, chemotherapy-induced heart damage, and/or a chemotherapy-induced reduction in cardiac function as a result of treatment with an anthracycline, optionally doxorubicin.

In some embodiments, the genotoxic stress-induced cardiac toxicity, the chemotherapy-induced cardiac toxicity, the chemotherapy-induced heart damage, and/or the chemotherapy-induced reduction in cardiac function is characterized by a reduction in cardiac contractility, a thinning of a ventricular wall, a reduction in cardiomyocyte size, or any combination thereof.

Alternatively or in addition, in some embodiments the genotoxic stress-induced cardiac toxicity, the chemotherapy-induced cardiac toxicity, the chemotherapy-induced heart damage, and/or the chemotherapy-induced reduction in cardiac function is associated with an inflammatory response, optionally wherein the inflammatory response comprises an induction of one or more of interleukin-1β (IL1β), interleukin-6 (IL-6), and tumor necrosis factor (Tnf). In some embodiments and because neutrophils are good sources of multiple chemokines and cytokines, various anti-inflammatory compounds (including but not limited to inflammasome inhibitors (e.g., that produce IL-1β).

In some embodiments, the genotoxic stress-induced cardiac toxicity results from undesirable migration to and infiltration of the cardiac muscle with neutrophils, particularly neutrophils that are undesirable activated. As a result, in some embodiments the methods of the presently disclosed subject matter employ compounds that are designed to inhibit neutrophil activation and/or inhibit neutrophil migration, particularly to the heart. Any compound that, when administered to a subject in need thereof inhibits neutrophil activation and/or neutrophil migration can be employed in the compositions and methods of the presently disclosed subject matter. Exemplary such compounds include CXCR2 inhibitors, such as but not limited to the pharmacological CXCR2 inhibitors SB225002 (N-(2-Hydroxy-4-nitrophenyl)-N′-(2-bromophenyl)urea) and SB265610 ((1-(2-Bromophenyl)-3-(4-cyano-1H-benzo[d] [1,2,3]triazol-7-yl)urea, N-(2-Bromophenyl)-N′-(7-cyano-1H-benzotriazol-4-yl)urea) and derivatives thereof, and repertaxin (R(−)-2-(4-isobutylphenyl)propionyl methansulphonamide). Other CXCR2 inhibitors are disclosed, for example, in Reissue U.S. Pat. Nos. RE47415 and RE47740, and U.S. Pat. Nos. 8,299,019; 10,301,378; and 10,370,363, each of which is incorporated herein by reference in its entirety.

In some embodiments, an inhibitor of the presently disclosed subject matter comprises, consists essentially of, or consists of a neutrophil myeloperoxidase inhibitor (MPOI). Exemplary MOIs include those disclosed in U.S. Pat. No. 11,246,870 (incorporated herein by reference in its entirety) and 2-thioxanthine (AZM198; see Antonelou et al., 2020). See also Tecchio et al., 2014 for a comprehensive listing of cytokines and chemokines that neutrophils do and/or can express.

Thus, in some embodiments the presently disclosed subject matter relates to compositions that comprise, consist essentially of, or consists of inhibitors of genotoxic stress-induced cardiac toxicity. As used herein the term “inhibit” refers to the ability of a compound or any agent to reduce and/or impede a described function and/or pathway. For example, inhibition can be by at least 10%, by at least 25%, by at least 50%, by at least 75%, by at least 80%, by at least 85%, by at least 90%, by at least 95%, by at least 97%, by at least 99%, or more.

A compound or agent that is capable of reducing and/or impeding a described function and/or pathway is thus an “inhibitor” of that function and/or pathway. Similarly, if the described function and/or pathway is associated with a predisposition to and/or development and/or worsening of any disease, disorder, and/or condition and/or any symptom and/or consequence thereof, the inhibitor can also function as an inhibitor of the disease, disorder, and/or condition and/or the symptom and/or consequence thereof. In the context of a disease, disorder, and/or condition and/or the symptom and/or consequence thereof, it is understood that the inhibition can be temporary or incomplete. In such embodiments, the inhibitor might not completely prevent the initiation, development, and/or progression of the disease, disorder, and/or condition and/or the symptom and/or consequence thereof but might delay its onset and/or slow the rate at which it develops and/or progresses. Thus, just as the degree to which the inhibitor might reduce and/or impede a biological function and/or pathway can be less than 100%, the degree to which the inhibitor delays or prevents the initiation, development, and/or progression of the disease, disorder, and/or condition and/or the symptom and/or consequence thereof and/or slows a rate at which it develops and/or progresses can be less than 100%. In some embodiments, a compound or agent is considered an inhibitor if its use results in any measurable improvement in the initiation, development, and/or progression of a disease, disorder, and/or condition and/or at least one symptom and/or consequence thereof, including but not limited to reducing a rate at which such a one symptom and/or consequence of the disease, disorder, and/or condition develops and/or progresses,

III.A. Pharmaceutical Compositions

In some embodiments, the compositions of the presently disclosed subject matter are provided as part of a pharmaceutical composition. As used herein, the term “pharmaceutical composition” refers to a composition comprising at least one active ingredient (e.g., an inhibitor of the presently disclosed subject matter), whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

In some embodiments, a pharmaceutical composition of the presently disclosed subject matter comprises, consists essentially of, or consists of at least one active ingredient (e.g., an inhibitor of the presently disclosed subject matter) and a pharmaceutically acceptable diluent and/or excipient. As used herein, the term “pharmaceutically acceptable” refers to physiologically tolerable, for either human or veterinary application. Similarly, “pharmaceutical compositions” include formulations for human and veterinary use. The term “pharmaceutically acceptable carrier” also refers to a chemical composition with which an appropriate compound or derivative can be combined and which, following the combination, can be used to administer the appropriate compound to a subject. In some embodiments, a pharmaceutically acceptable diluent and/or excipient is pharmaceutically acceptable for use in a human.

In some embodiments, the pharmaceutical compositions of the presently disclosed subject matter are for use in preventing and/or treating a disease or disorder associated with genotoxic stress-induced cardiac toxicity in a subject in need thereof.

The pharmaceutical compositions of the presently disclosed subject matter can in some embodiments consist of the active ingredient alone, in a form suitable for administration to a subject, or the pharmaceutical composition can in some embodiments comprise or consist essentially of the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt refers to an ester or salt form of the active ingredient which is compatible with any other ingredients of the pharmaceutical composition, which is not deleterious to the subject to which the composition is to be administered.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts.

III.A.1.Formulations

The compositions of the presently disclosed subject matter thus comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

Controlled- or sustained-release formulations of a pharmaceutical composition of the presently disclosed subject matter can be made using conventional technology. A formulation of a pharmaceutical composition of the invention suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin.

Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methy lcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl parahydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion.

The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter may also be prepared, packaged, or sold in a formulation suitable for parenteral administration, including but not limited to intraocular injection.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane dial, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems.

Compositions for sustained release or implantation may comprise pharmaceutically acceptable polymeric or hydrophobic materials such as an emulsion, an ion exchange resin, a sparingly soluble polymer, or a sparingly soluble salt. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, can in some embodiments have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985) Remington's Pharmaceutical Sciences, Mack Publishing Co., Easton, Pennsylvania, United States of America, which is incorporated herein by reference in itsz entirety.

III.A.2.Administration

With regard to administering a composition of the presently disclosed subject matter, methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intravitreous administration, including via intravitreous sustained drug delivery device, intracameral (into anterior chamber) administration, suprachoroidal injection, subretinal administration, subconjunctival injection, sub-tenon administration, peribulbar administration, transscleral drug delivery, intraocular injection, intravenous injection, intraparenchymal/intracranial injection, intra-articular injection, retrograde ureteral infusion, intrauterine injection, intratesticular tubule injection, intrathecal injection, intraventricular (e.g., inside cerebral ventricles) administration, administration via topical eye drops, and the like. Administration can be continuous or intermittent. In some embodiments, a preparation can be administered therapeutically; that is, administered to treat an existing disease or condition. In some embodiments, a preparation can be administered prophylactically; that is, administered for prevention of a disease, disorder, or condition.

III.A.3.Dose

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

In some embodiments, the presently disclosed subject matter also relates to methods for predicting heart failure in subjects previously exposed to a genotoxic agent, optionally a chemotherapeutic, further optionally where in the chemotherapeutic comprises an anthracycline, further optionally wherein the anthracycline is doxorubicin, the method comprising detecting the presence of therapy-related clonal hematopoiesis (t-CH) in the subject, wherein the presence of t-CH in the subject is predictive of heart failure in the subject. Methods for detecting the presence of therapy-related clonal hematopoiesis (t-CH) in subjects include those disclosed herein. Additional methods are disclosed in U.S. Pat. No. 10,041,044, which is incorporated herein by reference in its entirety.

The presently disclosed subject matter also related in some embodiments to uses of the compositions disclosed herein for treating or preventing genotoxic stress-induced cardiac toxicity, chemotherapy-induced cardiac toxicity, chemotherapy-induced heart damage, or chemotherapy-induced reduction in cardiac function. A such, in some embodiments the compositions for use of the presently disclosed subject matter comprise, consist essentially of, or consist of an effective amount of an inhibitor of neutrophil activation, an inhibitor of neutrophil migration, an inhibitor of neutrophil extracellular trap formation, an inhibitor of neutrophil cytokine and/or chemokine production, or any combination thereof.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

Without further description, it is believed that one of ordinary skill in the art can, using the preceding description and the following illustrative Examples, make and utilize the compounds of the presently disclosed subject matter and practice the methods of the presently disclosed subject matter. The following Examples therefore particularly point out embodiments of the presently disclosed subject matter and are not to be construed as limiting in any way the remainder of the disclosure.

### Materials and Methods for the EXAMPLES

Study approval: All procedures involving animal subjects have been approved by the Institutional Animal Care and Use Committee (IACUC) at the University of Virginia, Charlottesville, Virginia, United States of America.

Mice. Wild-type mice (Cd45.2), Trp53-insufficient mice, Trp53^{R27OH} mice, Trp53-floxed mice, Lyz2-Cre mice, Pep Boy mice (Cd45.1), and Ccr2^{gfp/gfp} mice were obtained from The Jackson Laboratory (stock 000664, 002103, 008182, 008462, 004781, 002014, and 027619, respectively). All strains are C57BL/6J background. Mice with myeloid-restricted Trp53 ablation were generated by crossing Trp53-floxed mice (Trp53^{fl/fl}) with Lyz2-Cre mice. Male mice were used for both in vivo and in vitro experiments unless otherwise noted. Mice were housed in a specific pathogen-free animal facility and given food and water ad libitum on 12-hour dark/light schedule.

Non-myeloablative bone marrow transplantation. 8-12 week old C57BL/6J Cd45.1 Pep Boy mice were transplanted with suspensions of bone marrow cells from two patterns of donors: 1) either Trp53-heterozygous mice or wild type mice (Trp53^{+/-} and Trp53^{+/+}); 2) either Trp53^{R27OH} mice or wild type mice (Trp53^{R27OH/+} and Trp53^{+/+}). 5×10⁶ of unfractionated bone marrow cells were injected to non-irradiated recipients via retro-orbital vein over consecutive 3 days (1.5×10⁷ cells in total) as described in (Wang et al., 2020).

Myeloablative bone marrow transplantation. 8-12 week old lethally-irradiated C57BL/6 Cd45.1 Pep Boy recipients were transplanted with suspensions of bone marrow cells containing 30% Cd45.2 Trp53^{+/-} cells and 70% Cd45.1 Trp53^{+/+} cells (30% Het mice), or 30% Cd45.2 Trp53^{+/+} cells and 70% Cd45.1 Trp53^{+/+} cells (30% Wild-type mice). Bone marrow cells were isolated from femurs and tibias of donor 8-12 week old mice after euthanasia. Recipient mice were irradiated in a pie cage (catalog IRD-P R; Braintree Scientific, Inc. Braintree, Massachusetts, United States of America) to limit mobility and ensure an equal dose of irradiation and were exposed to two radiation doses of 5.5 Gy four hours apart using the Cesium-137 irradiator (J.L. Shepherd & Associates, San Fernando, California, United States of America; 11 Gy in total). After the second irradiation, each recipient mouse was injected with 5×10⁶ bone marrow cells via the retro-orbital vein plexus. Sterilized caging, food and water were provided during the first 14 days post-transplantation, and water was supplemented with an antibiotic (Sulfatrim brand sulfamethoxazole and trimethoprim oral suspension; Teva, catalog 00703-9526-01).

Doxorubicin administration. Pattern 1: adoptive BMT mice were injected with doxorubicin 6 weeks after BMT using a model of therapy-related clonal hematopoiesis (Wang et al., 2020) involving 3 rounds of 2 mg/kg i.p. injection, with 3 weeks between each round. Pattern 2: competitive BMT mice, Trp53-Myelo-KO mice, Ccr2^{gfp/gfp} mice, and mice in neutrophil depletion and neutrophil recruitment inhibition studies were injected with doxorubicin with a dose of 15 μg/g (body weight) split into 3 injections at indicated time points. Pattern 3: wild-type mice were injected with a single dose of doxorubicin (15 μg/g) at indicated time points.

Monoclonal anti-mouse Ly6G antibody administration. This antibody was used to deplete neutrophils in vivo as described in Wang et al., 2019. Briefly, mice were injected intraperitoneally with 500 μg/injection of anti-Ly6G antibody every three days over a time course starting from 2 days before the start of doxorubicin administration until the end of the study. Isotype control anti-Trinitrophenol antibody (rat IgG2a) were similarly injected into a control group of mice. Antibody against Ly6G (clone 1A8) and rat IgG2 control antibody (clone 2A3) were purchased from BioXcell (Catalog Nos. BP0075-1 and BP0089, respectively). These antibodies were stored at 4° C. and the solution were mixed with PBS before injection (total volume of 200 μl). Injections were performed using a sterile syringe and 29-gauge needle. The expiration date of the mixtures coincided with the expiration date of the antibodies.

SB265610 (CXCR2 inhibitor) administration. SB265610 (1-(2-Bromophenyl)-3-(4-cyano-1H-benzo[d] [1,2,3]triazol-7-yl)urea, N-(2-Bromophenyl)-N′-(7-cyano-1H-benzotriazol-4-yl)urea; Catalog No. SML0421; Sigma-Aldrich, St. Louis, Missouri, United States of America) was used to inhibit neutrophil infiltration into tissues. Mice were injected intraperitoneally with 2 mg/kg of SB265610, once a day, over a time course that starts 1 day before the start of doxorubicin administration until the end of the study. The solution of SB265610 was prepared in sterile dimethyl sulfoxide (DMSO) to a concentration of 10 mg/ml. The aliquoted mixture was stored at −20° C. and thawed before use. The solution was mixed with corn oil (Sigma Catalog No. C8267) to minimize the toxic effect of DMSO just before injection. Injections were performed using a sterile syringe and needle (29G). The expiration date of the mixture coincided with the expiration date of the SB265610.

Echocardiography. Cardiac function was assessed before and after Dox administration at indicated time points using a Vevo 2100 ultrasound system equipped with MS400D probe (FUJIFILM VisualSonics Inc., Toronto, Ontario, Canada). Echocardiography was performed as described in Wang et al., 2019. Briefly, mice were anesthetized with isoflurane at a concentration of 5% (induction phase) and 1-1.5% (maintenance phase). Each animal was placed on the heating table in a supine position with the extremities taped to the table through four electrocardiography leads. Chest fur was removed with a chemical hair remover, and ultrasound gel was applied to the thorax surface to improve the visibility of the cardiac chambers. Left ventricular diameter at end-diastole (LVDd, mm), left ventricular diameter at end-systole (LVDs, mm), posterior wall thickness in diastole (PWTd, mm), and fractional shortening (FS, %) were measured from M-mode images obtained by short-axis view visualizing both mid papillary muscles. Measurements and analysis were performed by two individuals who were blinded to the identity of the experimental groups of mice using a Vevo Lab software (FUJIFILM VisualSonics Inc.).

Hematopoietic cell parameter measurements. Peripheral blood cells were obtained from the retro-orbital vein and collected in EDTA-coated tubes (BD Bioscicences, San Jose, California, United States of America). Hematopoietic parameters were analyzed using an Element HT5 Veterinary Hematology Analyzer (Heska, Loveland, Colorado, United States of America).

Flow Cytometry. Flow cytometry analysis of bone marrow cells, peripheral blood cells, and cardiac immune cells were performed as reported previously (see e.g. Sano et al., 2019; Wang et al., 2019; and Wang et al., 2020). The antibodies used for flow cytometric analysis are as described in Sano et al., 2019; Wang et al., 2019; and Wang et al., 2020. Bone marrow: Bone marrow cells were flushed out from 1 femur and 1 tibia, and red blood cells were lysed with RBC lysis buffer (Catalog No. 420301; BIOLEGEND®, San Diego, California, United States of America) for 20 seconds at room temperature. Dead cell staining was performed using ZOMBIE AQUA™ brand Fixable Viability Kit (Catalog No. 423102; BIOLEGEND®) in PBS for 15 minutes at room temperature. Cells were labeled with biotin-conjugated anti-mouse antibodies, and followed by incubation with BV650-Streptavidin (Catalog No. 405231; BIOLEGEND®) together with fluorochrome-conjugated antibodies for 20 minutes respectively at room temperature. Peripheral blood: Peripheral blood cells were obtained from the retro-orbital vein. Red blood cells were lysed with EBIOSCIENCE™ 1×RBC Lysis Buffer (Catalog No. 00-4333-57; Thermo Fisher Scientific, Waltham, Massachusetts, United States of America) for 5 minutes on ice. Incubation with antibodies was done for 20 minutes at room temperature in the dark. Heart tissue: Hearts were flushed with 15 ml of cold PBS from apex, and right atrium was excised to facilitate perfusion. Right ventricles and atriums were removed, and left ventricles were minced and digested in an enzyme cocktail: 450 U/ml collagenase I, 125 U/ml collagenase XI, 450 U/ml Hyaluronidase, and 60 U/ml DNase I (Catalog Nos. C0130, C7657, H3506, and D4513, respectively; Sigma-Aldrich), with the setting of 900 rpm, 37° C., 30 minutes in THERMOMIXER® C (Eppendorf North America, Enfield, Connecticut, United States of America). Hearts were subsequently homogenized through a 70 μm cell strainer (Catalog No. 352350; Thermo Fisher Scientific,).

Viable cells were identified as unstained cells with the ZOMBIE AQUA™ brand Fixable Viability Kit. Incubation with antibodies was done for 20 minutes at room temperature in the dark. 123COUNT EBEADS™ brand flow cytometry counting beads (Catalog No. 01-1234-42; Thermo Fisher Scientific) were used for quantification of absolute cell numbers. BD LSRII Flow Cytometer or Fortessa (BD Bioscience) was used for data acquisition. Data were analyzed with FlowJo Software. (FlowJo, LLC, Ashland, Oregon, United States of America).

Quantitative RT-PCR. Mouse total RNA was isolated using QIAzol reagent (Catalog No. 79306; Qiagen, Inc., Germantown, Maryland, United States of America), and was purified on the QIAcube. 1 μg RNA was reverse transcribed with a High-capacity cDNA Reverse Transcription Kit (Catalog No. 4368814; Thermo Fisher Scientific). qRT-PCR was performed with POWERUP™ SYBR™ Green reagent (Catalog No. 4367659; Thermo Fisher Scientific) in a QUANTSTUDIO™ 6 Flex PCR system (Thermo Fisher Scientific). Primers for gene expression studies are shown in the Table 2. Gene expression was calculated relative to 36b4 and normalized to controls. Data were calculated by AACt approach.

TABLE 2
Mus musculus qPCR Primer Sequences Employed

| Gene | Forward (5′-3′) | Reverse (5′-3′) |
|---|---|---|
| Nppa | AAGAACCTGCTAGACCAC | TGCTTCCTCAGTCTGCTCAC |
| | CTG (SEQ ID NO: 1) | (SEQ ID NO: 2) |
| aMHC | GCAGCAGCCCAGTACCTC | GTCATCAGGCACGAAGCA |
| | (SEQ ID NO: 3) | (SEQ ID NO: 4) |
| bMHC | CCTCACATCTTCTCCATC | TTGGATGACCCTCTTAGTGT |
| | TCTG (SEQ ID NO: 5) | TG (SEQ ID NO: 6) |
| II1b | TGACAGTGATGAGAATGA | TTGGAAGCAGCCCTTCATCT |
| | CCTGTTC (SEQ ID NO: | (SEQ ID NO: 8) |
| | 7) | |
| I16 | GCTACCAAACTGGATATA | CCAGGTAGCTATGGTACTCC |
| | ATCAGGA (SEQ ID NO: | AGAA (SEQ ID NO: 10) |
| | 9) | |
| Tnf | CGGAGTCCGGCAGG | GCTGGGTAGAGAATGGATGA |
| | (SEQ ID NO: 11) | A (SEQ ID NO: 12) |
| 36b4 | GCTCCAAGCAGATGCAGC | CCGGATGTGAGGCAGCAG |
| | A (SEQ ID NO: 13) | (SEQ ID NO: 14) |

Histology. Nitrotyrosine staining of hearts. Mice were sacrificed and perfused with cold PBS from the left ventricular apex. Hearts were excised and fixed with 10% formalin at 4° C. overnight. Heart tissues were dehydrated, embedded in paraffin and sectioned at a thickness of 7 μm. All histological analysis was performed with sections at the level of the papillary muscles. Nitrotyrosine staining was performed as follows. After deparaffinization with xylene and rehydration through decreasing ethanol concentrations, slides were heating at 98° C. to induce antigen retrieval with citrate antigen retrieval buffer (pH 6.0; Catalog No. 93678; Abcam plc., Cambridge, Massachusetts, United States of America). After three wash with TBS plus 0.05% Tween 20 (TBS-T), slides were incubated for 1 hour with 5% normal goat serum (Catalog No. S26, EMD-Millipore, Burlington, Massachusetts, United States of America) to block non-specific antibody binding. Then, sections were incubated for 2 hours with AffiniPure Fab Fragment Goat Anti-Mouse IgG (H+L; Catalog No. 115-007-003, Jackson Immuno Research Laboratories, Inc., West Grove, Pennsylvania, United States of America) to inhibit endogenous mouse immunoglobulins. Then, slides were incubated overnight at 4° C. with mouse anti-nitrotyrosine antibody (1:100; Catalog No. sc-32757; Santa Cruz Biotechnology, Dallas, Texas, United States of America) following by 1 hour incubation with Alexa Fluor 488-labeled goat anti-mouse secondary antibody (Catalog No. 11001; Thermo Fisher Scientific). Sections were also counterstained with DAPI (Catalog No. D1306, Thermo Fisher Scientific) and images were captured at 20× using a “all in one” BZ-X710 fluorescent microscope (Keyence Corporation of America, Itasca, Illinois, United States of America). Immunofluorescence staining was quantified by using the Fiji-ImageJ software and the arithmetic mean of six images was calculated for each sample. As a negative control, hearts from doxorubicin treated mice were incubated with blocking buffer and secondary antibody, but not with the primary antibody. Picro Sirius/Fast Green staining of heart. Deparaffinized sections were stained with freshly prepared PBS-based staining buffer composed of 1.2%/w saturated aqueous picric acid, 0.1%/w Fast Green FCF and 0.1%/w DirectRed 80 (Catalog Nos. 197378, F7252, and 365548, respectively, from Sigma-Aldrich). Sections were incubated for 1 hour at room temperature and the images were analyzed by Fiji-image J software for quantification of collagen content. Myocardial fibrosis size was expressed as a percentage of total LV area. Myocyte cross-sectional area (CSA). To determine CSA, wheat germ agglutinin staining of the heart sections from each experimental group was performed using Alexa Fluor 594 conjugated-WGA (Catalog NO. Wi 1262; Life Technologies, Waltham, Massachusetts, United States of America). Quantification of cardiomyocyte CSA was performed by an operator which is blinded to mouse genotypes and used a computer-assisted morphometric analysis tool on a Keyence BZ-X710 microscope. On average, 80-100 round-shaped cardiomyocytes per section were randomly selected for CSA analysis. Capillary density measurement. Capillary density was quantified by staining with Alexa Fluor 488-conjugated GS-Isolectin IB4 (Invitrogen, Catalog No. I21411). Exact numbers of cardiomyocytes and capillaries in the same view were measured and the ratio #capillaries/#cardiomyocytes was calculated as indicative of capillary density. On average, 5 views per section were randomly selected for capillary density analysis.

Ultra-low input RNA sequencing. For RNA sequencing analysis of blood neutrophil (CD45.2⁺Ly6G⁺CD115⁻), mice peripheral blood was initially collected in EDTA-coated tubes. After lysis of red blood cells, cells were stained with CD45.2, Ly6G, CD115 monoclonal antibodies. DAPI was used to exclude dead cells. Sorting was performed on a FACSARIA™ Fusion Cell Sorter platform (BD Biosciences) with an 85 μm nozzle and flow pressure set to 45 psi. A total of 50,000 cells were sorted directly into 1 ml of Trizol (Qiagen). Samples were sent to GENEWIZ (South Plainfield, New Jersey, United States of America) and subjected to library construction and sequencing. The HiSeq4000 platform was used for sequencing to generate paired end data of 150 bp. Sequence alignment and quantitation was performed by Kallisto 0.44.0 (Bray et al., 2016). Mus musculus GRCm38 FASTA transcriptome file from Ensembl database was used as an index for alignment. Differential gene expression analysis was performed using different R-studio analysis packages. WebGestalt online tool was used for the identification of statistically overrepresented (enriched) pathways among the differentially expressed genes identified by RNA-Seq analysis. After submission of the gene list, overrepresentation analysis on biological processes was performed based on GO with the Mus musculus genome. To identify the most significantly enriched gene sets, redundancy reduction was done with “weighted set cover” method.

Statistical analyses. GraphPad Prism 8.0 (GraphPad Software, San Diego, United States of America) was used for statistical analyses of all the experiments. Data are shown as mean±SEM except for the box plots, in which the whisker extends from minimum to maximum. The Shapiro-Wilk normality test was used to analyze data normality. Statistical tests included unpaired, two-tailed Student's t test (with Welch correction when variance was unequal) for normally distributed data and Mann-Whitney U test for non-normally distributed data. For multiple comparisons, one-way ANOVA with post-hoc Tukey's test (normally distributed data) or Kruskal-Wallis H test with post hoc Dunn's test (non-normal distributed data) was performed. Data with more than one variable were evaluated by two-way ANOVA with post-hoc Tukey's tests. Sequential data were evaluated by 2-way repeated-measures ANOVA with post hoc Sidak or Tukey multiple-comparison tests. Results were considered significantly different at 0.05.

### Example 1

### Establishment and Characterization of Trp53 Heterozygous-Deficient Mice (Trp53^{+/-})

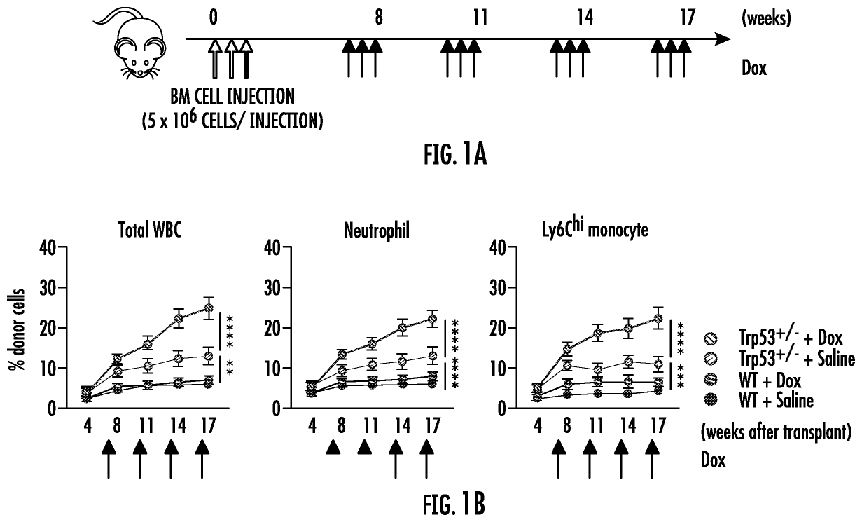

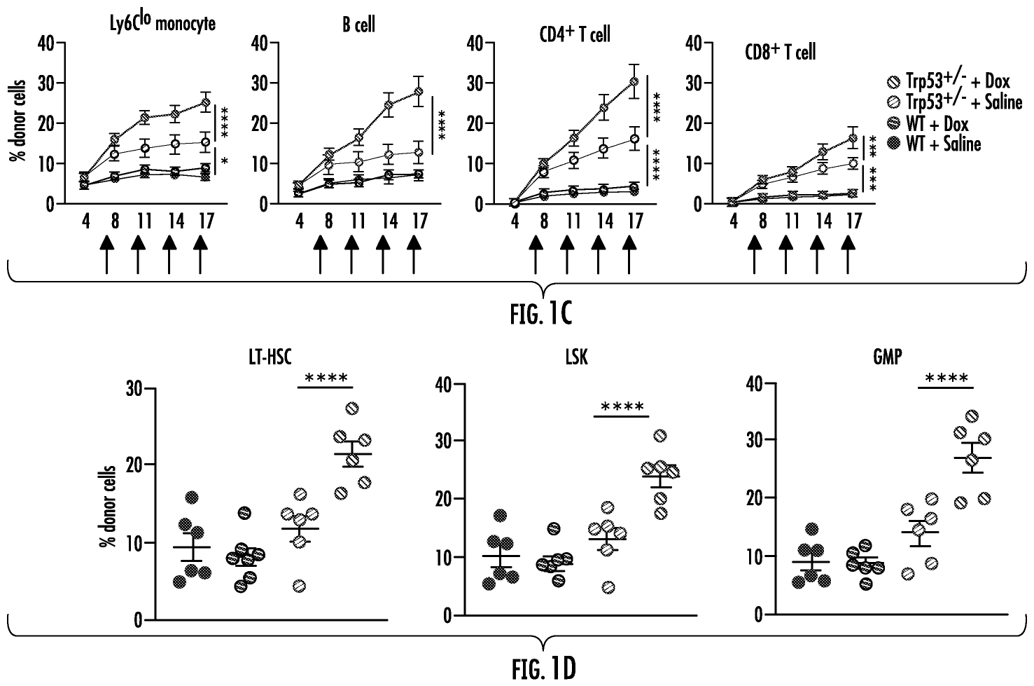

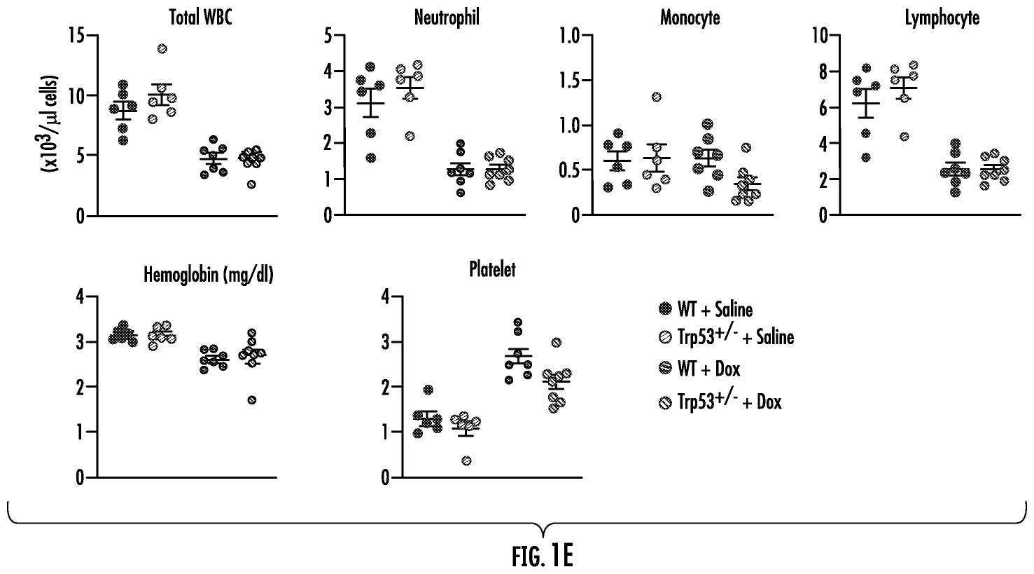

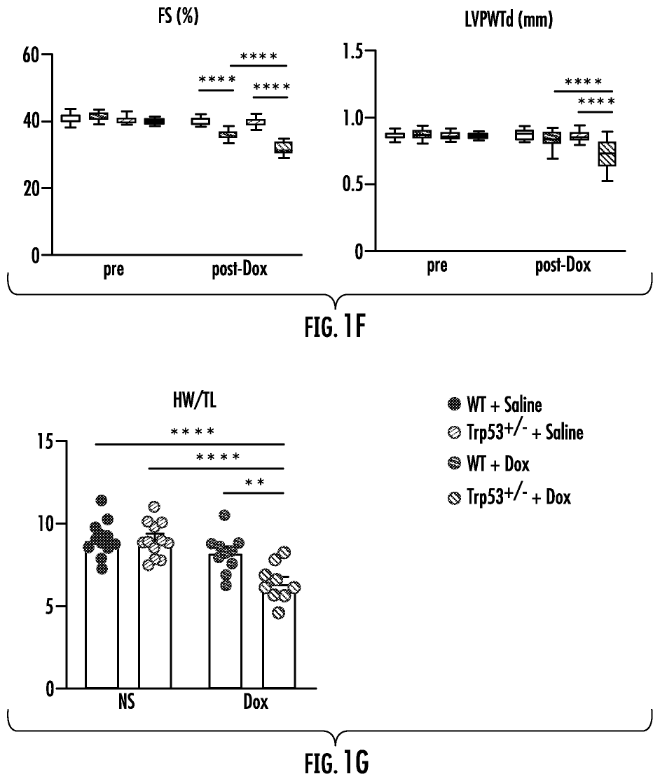

We established the animal model of clonal hematopoiesis in which a small proportion of HSPC harbor the Trp53 mutation. To overcome the possible limitations of myeloablative bone marrow transplant (BMT), we employed an adoptive transfer technique in which total bone marrow cells are injected to non-irradiated mice (FIG. 1A; Fuster et al., 2020; Wang et al., 2020). In this model, Trp53 heterozygous-deficient mice (Trp53^{+/-}) were used as donors to avoid hematologic malignancy and recapitulate the clinical state of clonal hematopoiesis in which one allele is typically mutated. Successful engraftment leading to the time-dependent expansion of the mutant HSPC in the unchallenged mice was achieved when a total of 1.5×10⁷ unfractionated BM cells were transferred intravenously to each recipient mouse on three consecutive days (FIG. 1). Mice were then treated with Dox or saline as control, administered in cycles, to examine the competitive fitness of Trp53^{+/-} HSPC under the conditions chemotherapeutic stress. The analysis of total white blood cells, neutrophils, and Ly6C′ monocytes in the control condition (saline) revealed that donor-derived Trp53^{+/-} cells achieved significantly higher levels of chimerism compared to wild-type cells over the 4 month time course, indicating a fitness advantage of Trp53^{+/-} clones under homeostatic conditions (FIG. 1). In support of the concept that TP53 is a driver of t-CH, Dox administration resulted in the further expansion of Trp53^{+/-} cells, but had little or no effect on the minimal expansion of transplanted wild-type cells when circulating cell populations were assessed (FIGS. 1B and 1C). In agreement with the observations in circulating leukocyte populations, the proportions of donor-derived Trp53^{+/-} cells in bone marrow lineage⁻Scal⁺cKit⁺ (LSK), lineage⁻Scal⁺cKit⁺CD48⁻CD150⁺ (long-term hematopoietic stem cell; LT-HSC), and lineage⁻Scal⁻cKit⁺CD16/32^{hi}CD34⁺ (granulocyte-monocyte progenitor; GMP) fractions were considerably higher under conditions of Dox treatment compared to the saline control at the termination of the experiment (FIG. 1D). Consistent with the paradigm of clonal hematopoiesis (Evans et al., 2020), there was no Trp53-dependent difference in hemoglobin levels or the absolute numbers of any hematopoietic cell type analyzed after the fourth cycle of Dox therapy, although the prolonged administration of Dox altered some of these parameters (FIG. 1E).

### Example 2

### Dox-Induced Clonal Expansion of Trp53 Mutants Participate in Cardiac Damage

To test whether the Dox-induced clonal expansion of Trp53 mutants participates in cardiac damage, we accessed cardiac function of these animals by echocardiography over the time course of the study. While mice transplanted with wild-type cells showed Dox-dependent deterioration of cardiac contractility and a thinning of the ventricular wall, mice with the expanding Trp53^{+/-} clones displayed significantly greater functional impairment and wall thinning (FIGS. 1F and 1G). Mice treated with Dox also displayed greater fibrosis and a reduced capillary density in the myocardium, and mice receiving the adoptive transfer of Trp53^{+/-} cells displayed a modestly augmented pathological response when assessed for these parameters.

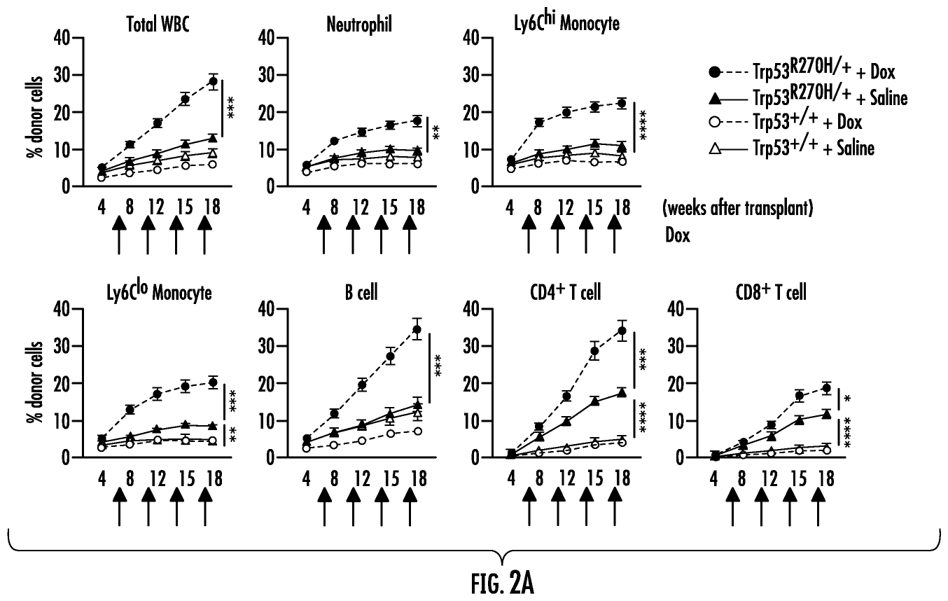

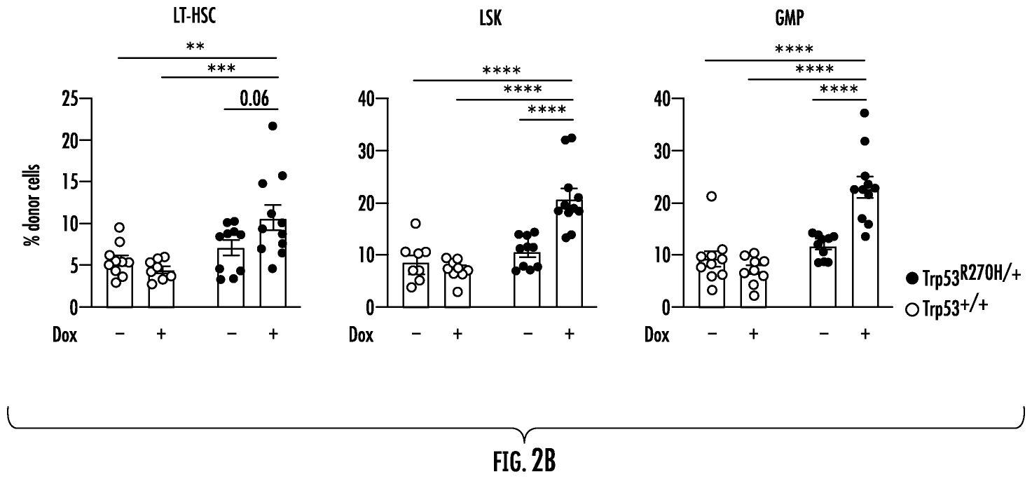

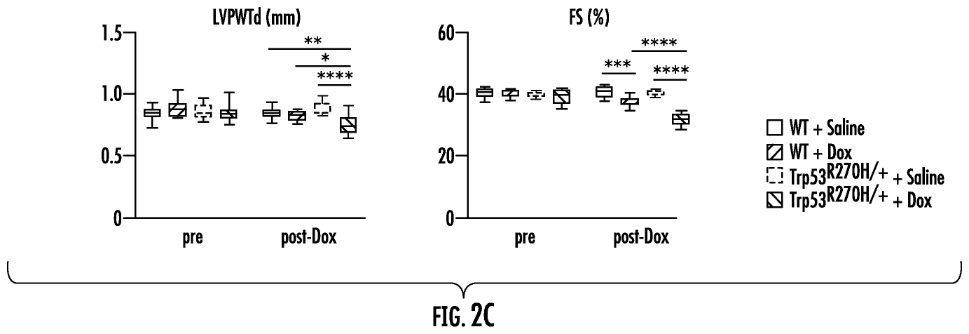

The vast majority of missense TP53 mutations in humans are mapped to its DNA-binding domain, and it has been suggested that mutants in this domain abrogate its sequence-specific DNA-binding activity (Coombs et al., 2017; Gibson et al., 2017; Boettcher et al., 2019). Thus, to recapitulate TP53-mediated clonal hematopoiesis under these conditions, we also analyzed the adoptive transfer bone marrow cells carrying Trp53^{R27OH} mutation (equivalent to the hotspot mutation R273H located at DNA-binding domain of human TP53). Donor-derived Trp53^{R27OH} clones exhibited a selective expansion in response to serial Dox administration as seen in circulating leukocyte populations and bone marrow HSPC fractions (FIGS. 2A and 2B). Mice harboring the expanding Trp53^{R27OH} mutant blood cells displayed a significant reduction in cardiac contractility as well as a wall thinning in response to the Dox therapy regimen when compared to mice transplanted with wild-type cells (FIG. 2C). Collectively, these results provide evidence supporting the concept that TP53-related clonal hematopoiesis promotes Dox-induced cardiotoxicity.

### Example 3

### Analysis of Wild-Type and or Trp53^{+/-} Bone Marrow Cells after Myeloablative BMT

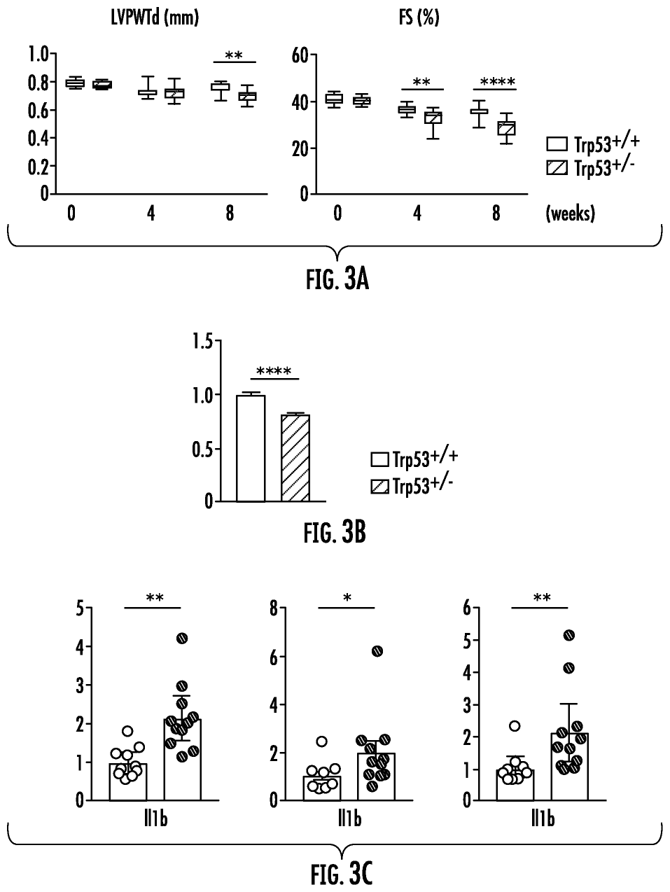

To obtain additional insights, we employed a myeloablative BMT strategy using donor wild-type or Trp53^{+/-} bone marrow cells adjusted to a variant allele fraction (VAF) of 0.15, that is typically observed in patient cohorts with t-CH (Coombs et al., 2017; Gibson et al., 2017; Bolton et al., 2018). Echocardiographic measurements revealed that while there were no differences between Trp53^{+/-} and wild-type groups before treatment, significant reductions of fractional shortening and posterior wall thickness of the heart were observed after the administration of Dox (FIG. 3A). Consistent with these data, histological measurements revealed reduced cardiomyocyte size, greater myocardial fibrosis, and diminished capillary density in the group with the Trp53 mutant cell condition (FIG. 3B). Transcript analysis of hearts at 8 weeks after Dox treatment showed significant increase in inflammatory mediators, including Il1b, Il6, and Tnf in mice transplanted with mutant Trp53 versus wild-type cells (FIG. 3C), suggesting that the Trp53Y mutant clones could be contributing to cardiotoxicity through an inflammatory mechanism.

### Example 4

### Analysis of the Role of the Immune System in Cardiotoxicity

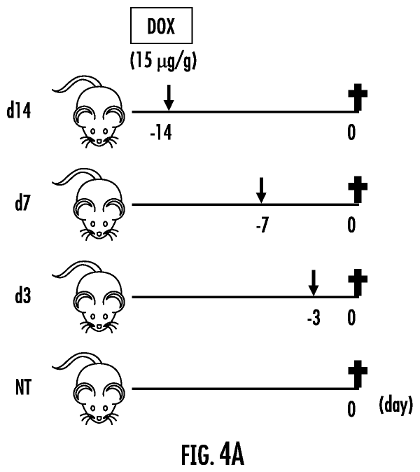

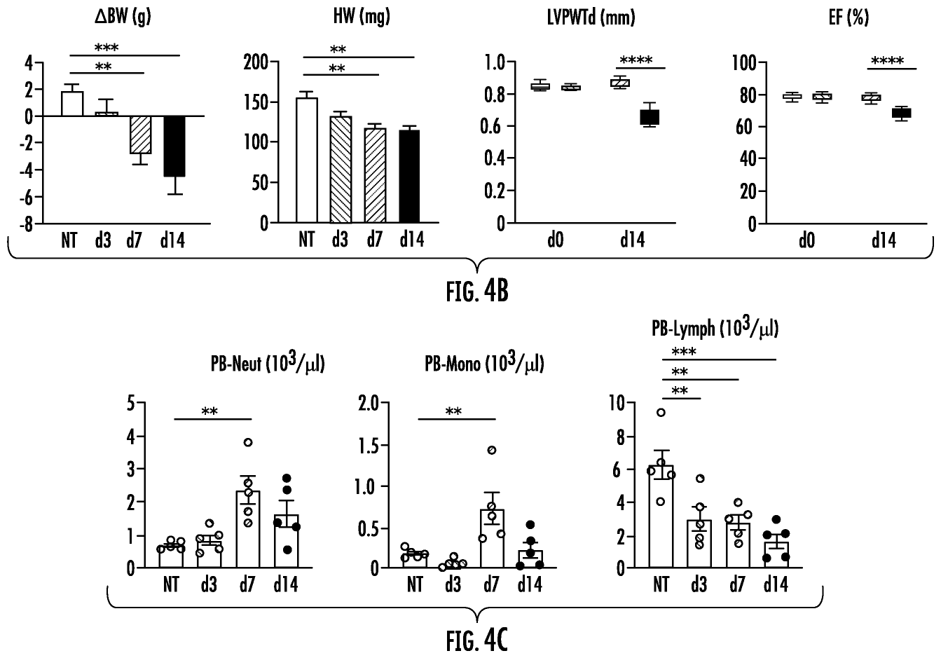

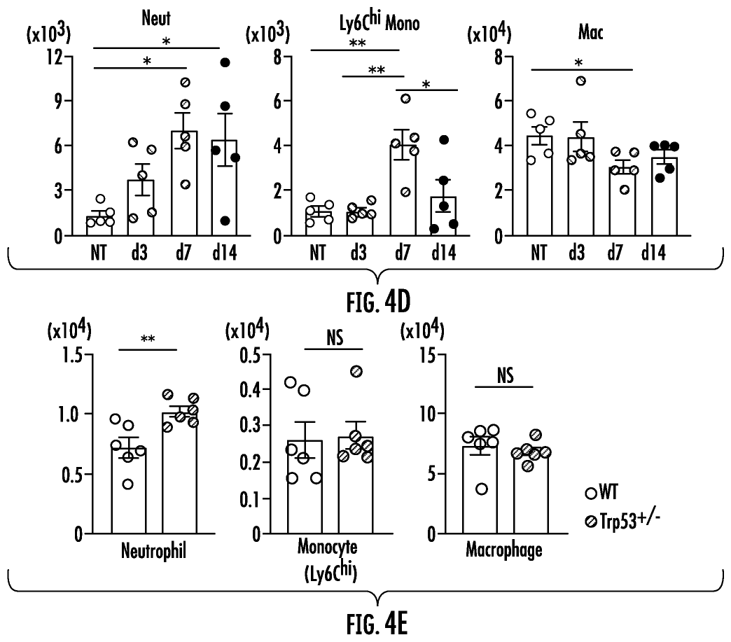

To further define the role of the immune system in Dox-mediated cardiotoxicity, wild-type mice were administered a single bolus of Dox and cardiac parameters, peripheral blood cell counts, and levels of cardiac-resident leukocytes were assessed at different time points (FIG. 4A). In this model, Dox administration led to the time-dependent loss of body weight and heart weight, and to reductions in cardiac wall dimension and function (FIG. 4B). These effects were accompanied by a transient increase of peripheral blood monocytes at the 7-day timepoint, and an increase in neutrophils at 7 days that was sustained until the termination of the experiment at 14 days (FIG. 4C). The analysis of cardiac-resident leukocytes revealed that monocyte number increased while macrophage number decreased, with a peak and nadir at the 7-day timepoint, respectively (FIG. 4D). Notably, Dox-induced neutrophil recruitment to the heart reached a peak at 7 days and levels were maintained until the termination of the experiment (FIG. 4D). This behavior contrasts with other models of cardiac injury that display a much more rapid and transient influx of neutrophils (Hilgendorf et al., 2014; Wang et al., 2019). Furthermore, mice that had been transplanted with Trp53 heterozygous mutant bone marrow cells displayed greater neutrophil recruitment at the 7 day time point compared to mice transplanted with wild-type cells, but treatment with Dox had no effect on the numbers of Ly6C^{hi} monocytes or macrophages within cardiac tissue under these conditions (FIG. 4E).

### Example 5

### Analysis of Trp53-Deficiency on Neutrophil Phenotype

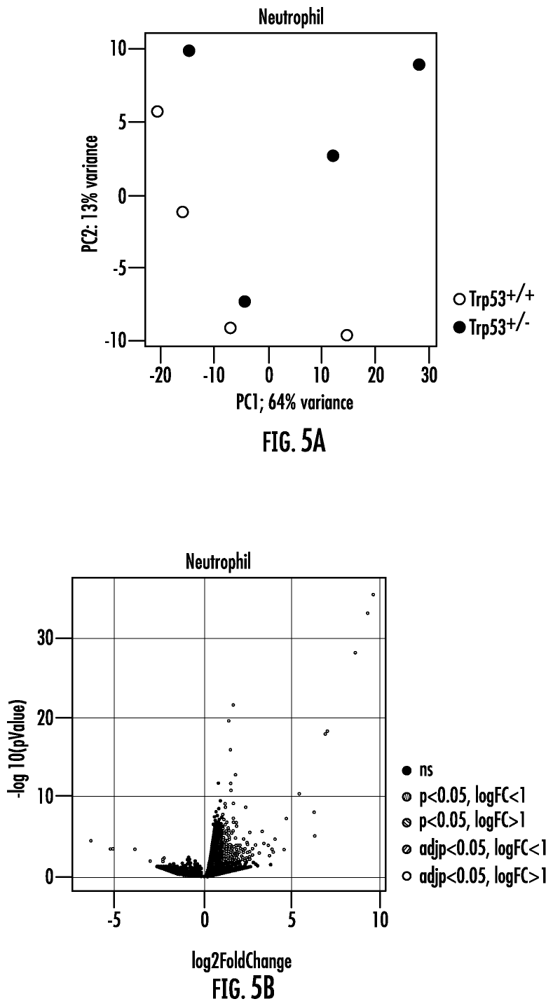

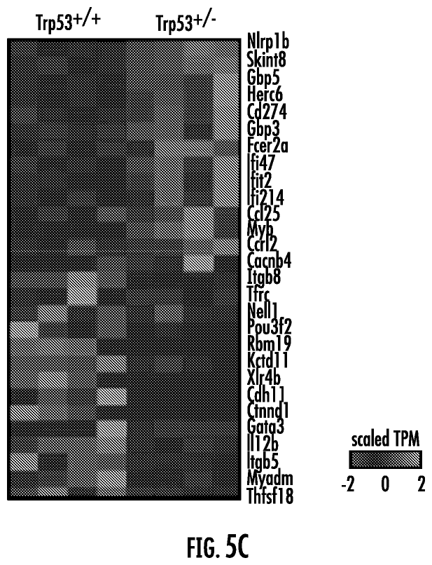

Recent studies have challenged the long-held view that neutrophils are transcriptionally silent (Silvestre-Roig et al., 2020). Thus, to evaluate the effect of Trp53-deficiency on neutrophil phenotype, RNA sequence analysis was performed on peripheral blood neutrophils from Trp53^{+/-} and wild-type mice at 1 day after Dox administration (Table 3). Principal component analysis revealed Trp53^{+/-} neutrophils have distinct gene expression profile from wild type neutrophils (FIG. 5A). With a cut off value of p<0.05 and TPM fold change>1.5, 898 genes were overexpressed and 63 genes were under expressed in Trp53^{+/-} neutrophils (FIG. 51B). Genes linked to immune response and cytokine response were significantly enriched in Trp53^{+/-} neutrophils among which are genes related to inflammasome pathway (Nlrp1b, Gbp5, Il18) and chemokines such as Ccl25, Ccrl2, and Cxcl1 (FIG. 5C and Table 4).

TABLE 3
Total reads after adapter trimming and Kallisto alignment.

| | Sample ID | Total processed reads | % aligned |
|---|---|---|---|
| Trp53^{+/+} | 4NWT1 | 75436734 | 84.5 |
| | 15LWT2 | 71971706 | 84.0 |
| | 17NWT3 | 82025052 | 84.8 |
| | 18RWT4 | 67990859 | 84.6 |
| Trp53^{+/-} | 13NHET1 | 105279399 | 83.1 |
| | 14RHET2 | 104355465 | 83.5 |
| | 19LHET3 | 96745097 | 86.1 |
| | 20RLHET4 | 89059415 | 80.4 |

TABLE 4
Over-representation Pathway Analysis
in Trp53-sufficient and Trp53-heterozygous
Insufficient Blood Neutrophils After Dox Administration*

| | No. of | | |
|---|---|---|---|
| | Genes | p Value | FDR |
| Enriched in Trp53-insufficient neutrophils | | | |
| Immune response | 75 | <0.05 | <0.05 |
| Defense response | 64 | <0.05 | <0.05 |
| Response to cytokine | 54 | <0.05 | <0.05 |
| Cell morphogenesis involved in neuron | 35 | <0.05 | <0.05 |
| differentiation | | | |
| Regulation of developmental growth | 26 | <0.05 | <0.05 |
| Enriched in Trp53-sufficient neutrophils | | | |
| Cell-cell adhesion | | <0.05 | >0.05 |
| Positive regulation of developmental process | | <0.05 | >0.05 |

*Top significant Gene Ontology (GO) biological terms related to upregulated 898 genes and downregulated 63 genes in the Trp53+/− blood neutrophils after Dox administration. Number of genes, p value, and FDR are shown.

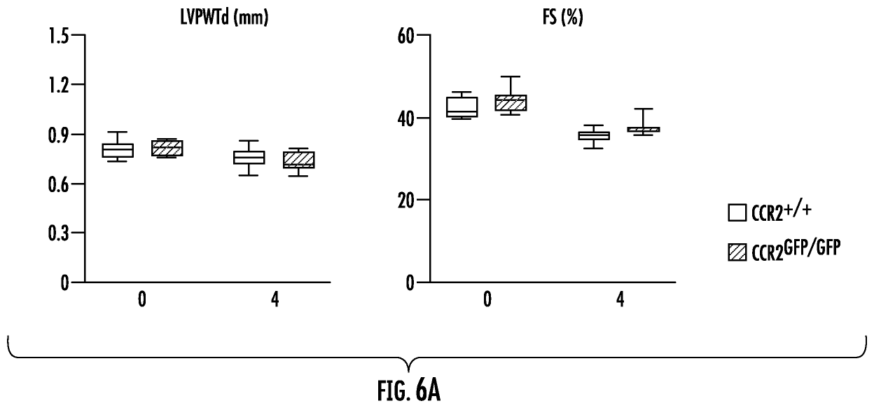

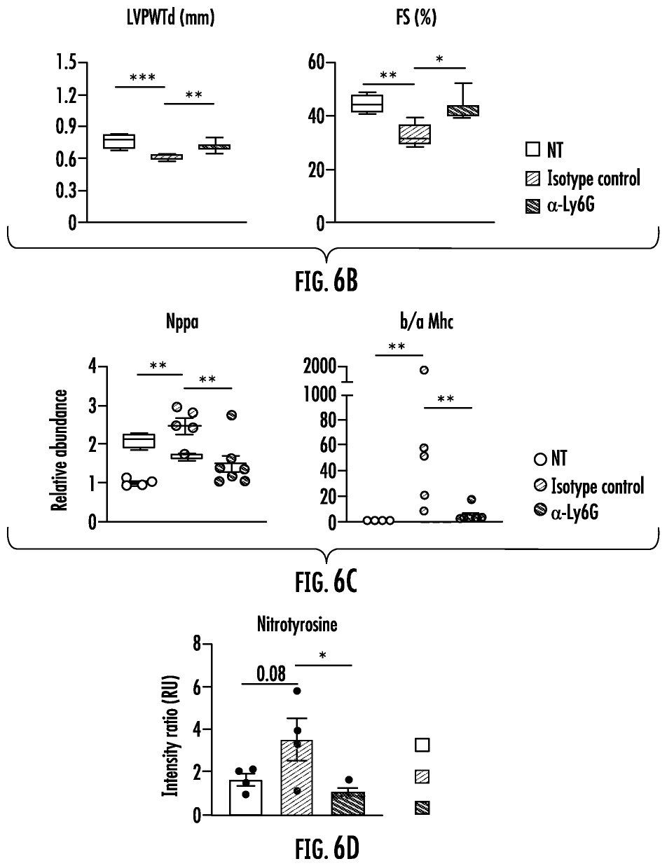

In view of these findings, experiments were performed to assess the roles of different myeloid cell populations in Dox-induced cardiac toxicity. Ccr2-deficient mice exhibit defects in monocyte egress from bone marrow (Dick et al., 2019). Consistently, the myocardium of Ccr2-deficient mice was largely devoid of CCR2⁺-infiltrating monocyte/macrophages compared with wild-type mice, but neutrophil content was not altered. Notably, Ccr2-deficient mice displayed comparable cardiac dysfunction after treatment with Dox relative to wild-type mice, suggesting little or no contribution of monocytes/macrophages to Dox-induced cardiac toxicity (FIG. 6A).

To examine the consequences of neutrophil infiltration in this model, wild-type mice were treated with Ly6G antibody to deplete neutrophils and Dox-induced cardiac toxicity was assessed. The Ly6G antibody largely protected against cardiac dysfunction as assessed by measures of posterior wall dimension and fractional shortening (FIG. 6B). Treatment with the Ly6G antibody also diminished the expression of the heart failure marker Nppa and the ratio of Mhc isoforms (FIG. 6C), and it reduced the level of oxidative stress in the myocardium as assessed by the accumulation of nitrotyrosine-protein adducts (FIG. 6D). In support of these findings, treatment with the pharmacological CXCR2 inhibitor SB265610, which functions to inhibit neutrophil trafficking, reduced neutrophil influx to the hearts of Dox-treated mice and reversed the effects of Dox on cardiac wall thinning and function (FIG. 6E). Collectively, these data suggested that neutrophil involvement was a significant component of Dox-mediated cardiac toxicity in wild-type mice.

### Example 6

### Further Analysis of Myeloid Cell Participation in Cardiac Toxicity

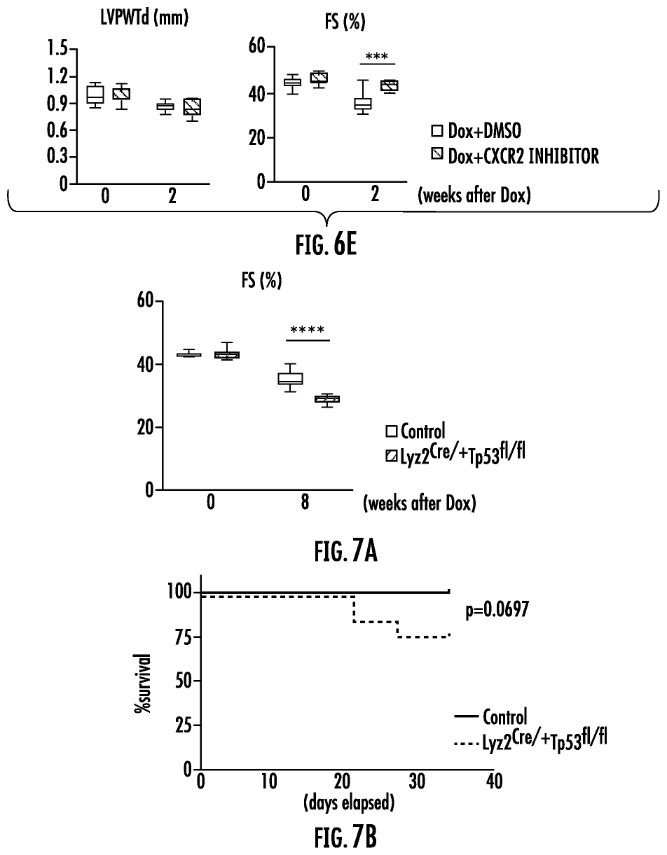

Further experiments examined myeloid cell participation in the amplified cardiac toxicity observed in the Trp53-mediated t-CH model. To this end, myeloid-specific Trp53-deficient mice (Lyz2^{Cre/+}Trp53^{fl/fl}) treated with Dox displayed marked reduction in cardiac function compared to control (Lyz2^{+/+}Trp53^{fl/f/}) mice (FIG. 7A). Notably, the Dox-induced cardiac toxicity in this strain was much more severe in this strain than in mice with partial Trp53-deficiency (compare with FIGS. 3A-3C), and this severe dysfunction was associated with a reduced survival compared to control mice treated with Dox (FIG. 7B) The myeloid-restricted Trp53-deficient mice also displayed upregulation of transcripts that encode inflammatory mediators in the myocardium compared to control mice (FIG. 7C).

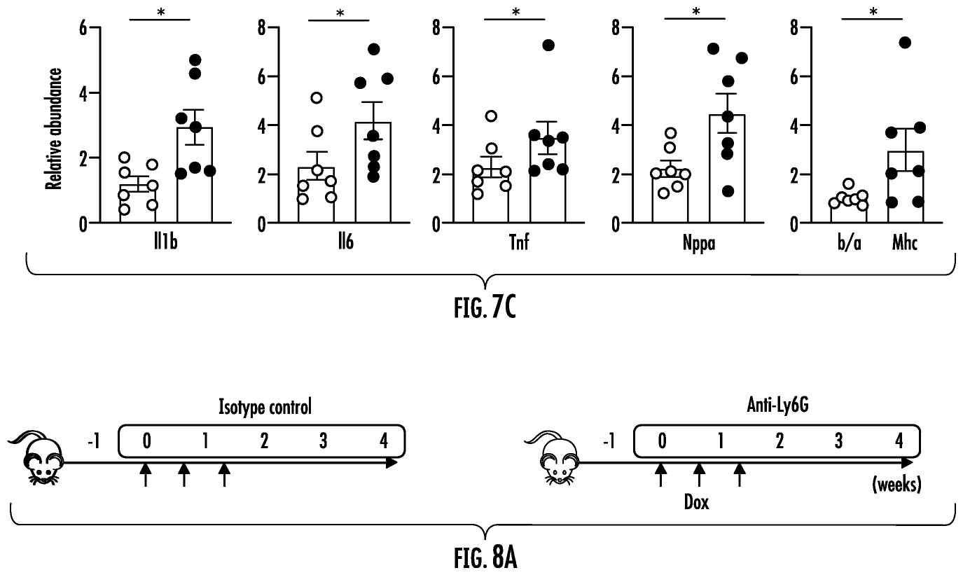

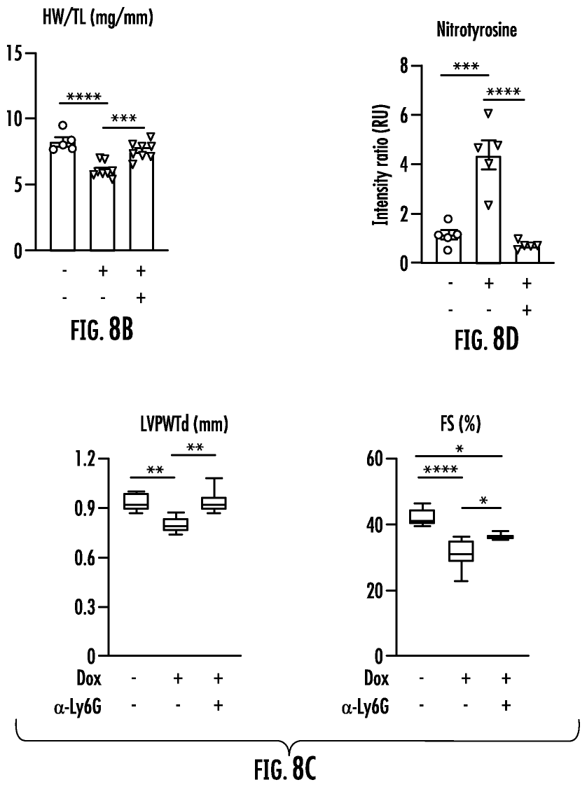

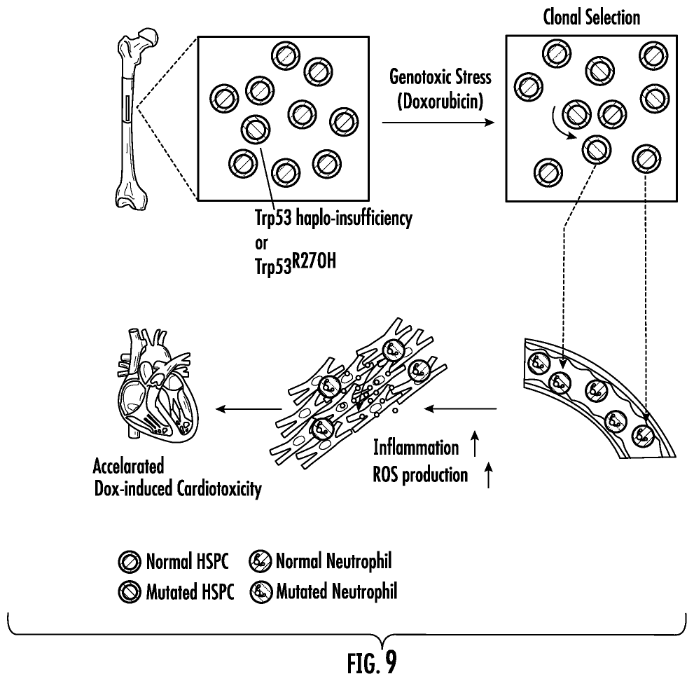

To investigate whether neutrophil involvement is essential for cardiac toxicity in the t-CH model, mice underwent BMT to establish a VAF of 0.15 with heterozygous Trp53-deficient cells and were then treated with a course of Dox or saline in the presence of anti-Ly6G antibody, to deplete neutrophils, or an isotype control antibody (FIG. 8A). Neutrophil depletion ameliorated the detrimental effects of Dox on heart weight and echocardiographic parameters in mice with the expanding Trp53-deficient HSPC (FIGS. 8B and 8C). Assessing nitrotyrosine-protein adducts in myocardial tissues from the different experimental groups of mice revealed that Trp53-deficient condition markedly augmented nitrotyrosine content and that neutrophil depletion with anti-Ly6G antibody reversed this damage (FIG. 8D).

### Discussion of the EXAMPLES

Dox and other anthracyclines are used to treat many solid and hematologic cancers. However, the clinical utility of this class of drugs is limited by their immediate and prolonged cardiac toxicity (Zamorano et al., 2016; Campia et al., 2019). Studies of anthracycline effects on the heart have largely focused on cardiac myocyte toxicity (Lipshultz et al., 2013; Vejpongsa & Yeh, 2014), while the potential role of immune cells in this condition has received less attention. These data revealed that Dox-induced cardiotoxicity involves an unexpected and complex interplay between the myocardium and the immune system. It has been shown that Dox interacts with topoisomerase II 0 leading to DNA double-strand breaks, mitochondrial dysfunction, elevations in oxidative and nitrosative stress, and death in cardiac myocytes (Zhang et al., 2012).

Disclosed herein is the discovery that that Dox therapy leads to the prolonged infiltration of neutrophils to the heart, and that neutrophil depletion or inhibition will diminish the cardiotoxic actions of Dox. Oxidative/nitrosative stress damage is a widely recognized component of Dox cardiac toxicity, and the experimental data provided herein suggest that infiltrating neutrophils are a significant source of this stress. Consistent with these experimental findings, clinical studies have found that anthracycline-induced heart failure is associated with elevated serum levels of neutrophil marker proteins and genetic variants that lead to neutrophil ROS/reactive nitrogen species generation (Wojnowski et al., 2005; Putt et al., 2015; Leong et al., 2017). While traditionally thought to largely participate in acute inflammatory responses, these data add to the growing realization that neutrophils can contribute to chronic disease processes (Talukdar et al., 2012; Silvestre-Roig et al., 2019).

The presently disclosure also provides evidence to suggest that t-CH is a factor that contributes to the cardiac toxicity that develops in cancer patients who are treated with anthracyclines, and that this effect operates at least in part by augmenting the infiltration and activation of neutrophils. Specifically, it is shown that Trp53-deficient HSPCs undergo rapid expansion in response to Dox treatment, and that the mutant neutrophil progeny of these HSPCs markedly augment the cardiotoxicity that results from Dox therapy. In Dox-treated mice, it was found that heterozygous Trp53-deficiency in neutrophils led to further elevations in myocardial oxidative/nitrosative stress and that Trp53-deficient neutrophils displayed numerous transcriptional changes, including the elevation of chemokine transcripts that promote neutrophil recruitment and activation. In light of these data, it is tempting to speculate that t-CH can impact both the acute and chronic effects of anthracyclines on the heart. As indicated by the results presented herein, Dox administration can lead to the rapid expansion of a preexisting TP53 clone that amplifies the cardiotoxicity of the Dox therapy. Furthermore, once these clones have undergone expansion, they can potentially exert chronic pathological actions in the myocardium of aging individuals who have been treated for cancer when they are exposed to other cardiovascular stresses. Collectively, these data suggest that t-CH can be predictive of heart failure in cancer survivors and that assessing the status of clonal hematopoiesis before and after cancer therapy could provide guidance for personalized therapies to protect the heart from the short and long-term effects of genotoxic drugs.

### REFERENCES

All references listed in the instant disclosure and in the Appendices attached hereto, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altschul et al. (1990a) Basic local alignment search tool. J Mol Biol 215:403-410.
Altschul et al. (1990b) Protein database searches for multiple alignments. Proc Natl Acad Sci USA 87:14:5509-13.
Altschul et al. (1997) Gapped BLAST and PSI-BLAST: a new generation of protein database search programs. Nucleic Acids Res 25:3389-3402.
Antonelou et al. (2020) Therapeutic Myeloperoxidase Inhibition Attenuates Neutrophil Activation, ANCA-Mediated Endothelial Damage, and Crescentic GN. JASN 31:350-364.
Boettcher et al. (2019) A dominant-negative effect drives selection of TP53 missense mutations in myeloid malignancies. Science 365:599-604.
Bolton et al. (2019) Oncologic therapy shapes the fitness landscape of clonal hematopoiesis. bioRxiv 848739 doi:10.1101/848739.
Bray et al. (2016) Near-optimal probabilistic RNA-seq quantification. Nat Biotechnol 34:525-527.
Campia et al. (2019) Cardio-Oncology: Vascular and Metabolic Perspectives: A Scientific Statement From the American Heart Association. Circulation 139:e579-e602.
Cardinale et al. (2015) Early detection of anthracycline cardiotoxicity and improvement with heart failure therapy. Circulation 131(22):1981-1988.
Coombs et al. (2017) Therapy-Related Clonal Hematopoiesis in Patients with Non-hematologic Cancers Is Common and Associated with Adverse Clinical Outcomes. Cell Stem Cell 21:374-382.e4.
Dick et al. (2019) Self-renewing resident cardiac macrophages limit adverse remodeling following myocardial infarction. Nat Immunol 20(1):29-39.
Dorsheimer et al. (2019) Association of Mutations Contributing to Clonal Hematopoiesis With Prognosis in Chronic Ischemic Heart Failure. JAMA Cardiol 4:25-33.
Evans et al. (2020) Cardiovascular Disease, Aging, and Clonal Hematopoiesis. Annu Rev Pathol Mech Dis 15:419-438.
Faber et al. (2018) Burden of cardiovascular risk factors and cardiovascular disease in childhood cancer survivors: data from the German CVSS-study. Eur Heart J 39(17):1555-1562.
Fidler et al. (2016) Long term cause specific mortality among 34 489 five year survivors of childhood cancer in Great Britain: population based cohort study. BMJ 354:i4351.
Fuster et al. (2017) Clonal hematopoiesis associated with TET2 deficiency accelerates atherosclerosis development in mice. Science 355:842-847.
Fuster et al. (2020) TET2-loss-of-function-driven clonal hematopoiesis exacerbates experimental insulin resistance in aging and obesity. Cell Rep 33(4):108326.
Genovese et al. (2014) Clonal hematopoiesis and blood-cancer risk inferred from blood DNA sequence. N Engl J Med 371:2477-2487.
Gibson et al. (2017) Clonal Hematopoiesis Associated With Adverse Outcomes After Autologous Stem-Cell Transplantation for Lymphoma. J Clin Oncol 35:1598-1605.
Gross & Mienhofer (eds.) (1981) The Peptides, Vol. 3, Academic Press, New York, New York, pp. 3-88.
Hilgendorf et al. (2014) Ly-6Chigh monocytes depend on Nr4a1 to balance both inflammatory and reparative phases in the infarcted myocardium. Circ Res 114:1611-1622.
Hsu et al. (2018) PPM1D Mutations Drive Clonal Hematopoiesis in Response to Cytotoxic Chemotherapy. Cell Stem Cell 23:700-713.e6.
Jaiswal et al. (2014) Age-related clonal hematopoiesis associated with adverse outcomes. N Engl J Med 371:2488-2498.
Jaiswal et al. (2017) Clonal Hematopoiesis and Risk of Atherosclerotic Cardiovascular Disease. N Engl J Med 377:111-121.
Jimenez et al. (1997) Dysregulated expression of neutrophil apoptosis in the systemic inflammatory response syndrome. Arch. Surg 132:1263-1269.
Kahn et al. (2018) PPM1D-truncating mutations confer resistance to chemotherapy and sensitivity to PPM1D inhibition in hematopoietic cells. Blood 132:1095-1105.
Karlin & Altschul (1990) Methods for assessing the statistical significance of molecular sequence features by using general scoring schemes. Proc Natl Acad Sci USA 87:2264-2268.
Karlin & Altschul (1993) Applications and statistics for multiple high-scoring segments in molecular sequences. Proc Natl Acad Sci USA 90:5873-5877.
Kilpatrick et al. (2002) A role for PKC-delta and PI 3-kinase in TNF-alpha-mediated antiapoptotic signaling in the human neutrophil. Am J Physiol Cell Physiol 283:C48-57.
Lee et al. (1993) Inhibition of apoptosis and prolongation of neutrophil functional longevity by inflammatory mediators. J Leukoc Biol 54:282-288.
Leong et al. (2017) Candidate Gene Association Studies of Anthracycline-induced Cardiotoxicity: A Systematic Review and Meta-analysis. Sci Rep 7:39.
Leslie (2020) The Body's Dangerous Defenders. Science 367(6482) 1067-1069.
Lipshultz et al. (2013) Long-term Cardiovascular Toxicity in Children, Adolescents, and Young Adults Who Receive Cancer Therapy: Pathophysiology, Course, Monitoring, Management, Prevention, and Research Directions: A Scientific Statement From the American Heart Association. Circulation 128:1927-1995.
Mas-Peiro et al. (2020) Clonal haematopoiesis in patients with degenerative aortic valve stenosis undergoing transcatheter aortic valve implantation. Eur Heart J 41:933-939.
Putt et al. (2015) Longitudinal Changes in Multiple Biomarkers Are Associated with Cardiotoxicity in Breast Cancer Patients Treated with Doxorubicin, Taxanes, and Trastuzumab. Clin Chem 61:1164-1172.
Reissue U.S. Pat. Nos. RE47415, RE47740.
Sano et al. (2018a) CRISPR-Mediated Gene Editing to Assess the Roles of Tet2 and Dnmt3a in Clonal Hematopoiesis and Cardiovascular Disease Circ Res 123:335-341.
Sano et al. (2018b) Tet2-Mediated Clonal Hematopoiesis Accelerates Heart Failure Through a Mechanism Involving the IL-1P/NLRP3 Inflammasome. J Am Coll Cardiol 71:875-886.
Sano et al. (2019) JAK2 (V617F)-Mediated Clonal Hematopoiesis Accelerates Pathological Remodeling in Murine Heart Failure. JACC Basic Transl Sci 4:684-697.
Silvestre-Roig et al. (2019) Externalized histone H4 orchestrates chronic inflammation by inducing lytic cell death. Nature 569:236-240.
Silvestre-Roig et al. (2020) Neutrophils as regulators of cardiovascular inflammation. Nat Rev Cardiol 17:327-340.
Stoltzfus et al. (2020) Fatal heart disease among cancer patients. Nat Commun 11(1):2011.
Talukdar et al. (2012) Neutrophils mediate insulin resistance in mice fed a high-fat diet through secreted elastase. Nat Med 18:1407-1412.
Taneja et al. (2004) Delayed neutrophil apoptosis in sepsis is associated with maintenance of mitochondrial transmembrane potential and reduced caspase-9 activity. Crit Care Med 32:1460-1469.
Tecchio et al. (2014) Neutrophil-derived cytokines: facts beyond expression. Front Immunol 5:508.
U.S. Pat. Nos. 8,299,019; 10,301,378; 10,370,363; 10,041,044; 11,246,870.
Vejpongsa & Yeh (2014) Prevention of anthracycline-induced cardiotoxicity: Challenges and opportunities. J Am Coll Cardiol 64:938-945.
Wang et al. (2019) Wnt5a-Mediated Neutrophil Recruitment Has an Obligatory Role in Pressure Overload-Induced Cardiac Dysfunction. Circulation 140:487-499.
Wang et al. (2020) Tet2-mediated clonal hematopoiesis in nonconditioned mice accelerates age-associated cardiac dysfunction. JCI Insight 5:e135204.
Wojnowski et al. (2005) NAD(P)H oxidase and multidrug resistance protein genetic polymorphisms are associated with doxorubicin-induced cardiotoxicity. Circulation 112:3754-3762.
Wolach et al. (2018) Increased neutrophil extracellular trap formation promotes thrombosis in myeloproliferative neoplasms. Sci Transl Med 10:eaan8292, doi: 10.1126/scitranslmed.aan8292.
Wong et al. (2015) Role of TP53 mutations in the origin and evolution of therapy-related acute myeloid leukaemia. Nature 518:552-555.
Yeh & Bickford (2009) Cardiovascular complications of cancer therapy: incidence, pathogenesis, diagnosis, and management. J Am Coll Cardiol 53(24):2231-2247.
Zamorano et al. (2016) 2016 ESC Position Paper on cancer treatments and cardiovascular toxicity developed under the auspices of the ESC Committee for Practice Guidelines: The Task Force for cancer treatments and cardiovascular toxicity of the European Society of Cardiology (ESC) Eur Heart J 37:2768-2801.
Zamorano et al. (2020) The cancer patient and cardiology. Eur J Heart Fail 22(12):2290-2309. et al. (2012) Identification of the molecular basis of doxorubicin-induced cardiotoxicity. Nat Med 18:1639-1642.

It will be understood that various details of the presently disclosed subject matter can be changed without departing from the scope of the presently disclosed subject matter. Furthermore, the foregoing description is for the purpose of illustration only, and not for the purpose of limitation.

1. A method for treating or preventing genotoxic stress-induced cardiac toxicity, the method comprising administering to a subject in need thereof an effective amount of an inhibitor of therapy-related clonal hematopoiesis (t-CH), neutrophil activation, neutrophil migration, neutrophil extracellular trap formation, neutrophil cytokine and/or chemokine production, or any combination thereof.

2. The method of claim 1, wherein the genotoxic stress-induced cardiac toxicity results from exposure to one or more anti-tumor and/or anti-cancer therapies, optionally wherein the anti-tumor and/or anti-cancer therapies comprise treatment with radiation and/or treatment with one or more chemotherapeutic agents and/or one or more immunomodulatory agents.

3. The method of claim 2, wherein the genotoxic stress-induced cardiac toxicity comprises chemotherapy-induced cardiac toxicity, chemotherapy-induced heart damage, and/or a chemotherapy-induced reduction in cardiac function.

4. The method of claim 3, wherein the subject in need thereof has or is at risk for developing chemotherapy-induced cardiac toxicity, chemotherapy-induced heart damage, and/or a chemotherapy-induced reduction in cardiac function as a result of treatment with an anthracycline, a platinum compound, a topoisomerase inhibitor, or any combination thereof.

5. The method of claim 4, wherein the anthracycline is aclarubicin, daunorubicin, epirubicin, mitoxantrone, valrubicin, or doxorubicin, optionally doxorubicin.

6. The method of claim 4, wherein the platinum compound is selected from the group consisting of cisplatin, carboplatin, oxaliplatin, nedaplatin, lobaplatin, triplatin tetranitrate, phenanthriplatin, picoplatin, and satraplatin, or any combination thereof.

7. The method of claim 4, wherein the topoisomerase inhibitor is selected from the group consisting of teniposide, irinotecan, etoposide, topotecan, mitoxantrone, moxifloxacin, grepafloxacin, dexrazoxane, valrubicin, and epirubicin, or any combination thereof.

8. The method of claim 4, wherein the genotoxic stress-induced cardiac toxicity, the chemotherapy-induced cardiac toxicity, the chemotherapy-induced heart damage, and/or the chemotherapy-induced reduction in cardiac function is characterized by a reduction in cardiac contractility, a thinning of a ventricular wall, a reduction in cardiomyocyte size, or any combination thereof.

9. The method of claim 4, wherein the genotoxic stress-induced cardiac toxicity, the chemotherapy-induced cardiac toxicity, the chemotherapy-induced heart damage, and/or the chemotherapy-induced reduction in cardiac function is associated with an inflammatory response.

10. The method of claim 9, wherein the inflammatory response comprises an induction of one or more of interleukin-1β (IL-1β), interleukin-6 (IL-6), and tumor necrosis factor (Tnf).

11. The method of claim 9, wherein the subject is a cancer survivor who is relatively young and/or does not have other known cardiovascular risk factors.

12. The method of claim 1, wherein the genotoxic stress-induced cardiac toxicity results from exposure to ionizing radiation.

13. The method of claim 1, wherein the genotoxic stress-induced cardiac toxicity results from exposure to a platinum compound, a topoisomerase inhibitor, or any combination thereof.

14. The method of claim 9, wherein the inhibitor of therapy-related clonal hematopoiesis (t-CH), neutrophil activation, neutrophil migration, neutrophil extracellular trap formation, neutrophil cytokine and/or chemokine production is selected from the group consisting of avacopan, danirixin, nemiralisib, lonodelestat, alvelestat, and elafin.

15. A method for predicting heart failure in a subject previously exposed to a genotoxic agent, the method comprising, consisting essentially of, or consisting of detecting the presence of therapy-related clonal hematopoiesis (t-CH) in the subject, wherein the presence of t-CH in the subject is predictive of heart failure in the subject.

16. The method of claim 15, wherein the genotoxic agent is a chemotherapeutic agent.

17. The method of claim 16, wherein in the chemotherapeutic comprises an anthracycline, a platinum compound, a topoisomerase inhibitor, or any combination thereof.

18. The method of claim 17, wherein the anthracycline is aclarubicin, daunorubicin, epirubicin, mitoxantrone, valrubicin, or doxorubicin, optionally doxorubicin.

19. The method of claim 17, wherein the platinum compound is selected from the group consisting of cisplatin, carboplatin, oxaliplatin, nedaplatin, lobaplatin, triplatin tetranitrate, phenanthriplatin, picoplatin, and satraplatin, or any combination thereof.

20. The method of claim 17, wherein the topoisomerase inhibitor is selected from the group consisting of teniposide, irinotecan, etoposide, topotecan, mitoxantrone, moxifloxacin, grepafloxacin, dexrazoxane, valrubicin, and epirubicin, or any combination thereof.

21. The method of claim 15, wherein the genotoxic agent comprises, consists essentially of, or consists of ionizing radiation.

22. The method of claim 15, wherein the subject is a mammal.

23. The method of claim 22, wherein the subject is a human.

24. Use of a composition comprising an inhibitor of therapy-related clonal hematopoiesis (t-CH), an inhibitor of neutrophil activation, an inhibitor of neutrophil migration, an inhibitor of neutrophil extracellular trap formation, an inhibitor of neutrophil cytokine and/or chemokine production, or any combination thereof for treating or preventing genotoxic stress-induced cardiac toxicity, chemotherapy-induced cardiac toxicity, chemotherapy-induced heart damage, or chemotherapy-induced reduction in cardiac function.


SEQUENCE LISTING (15 sequences)

SEQ ID NO: 1 - DNA, 21 bp, Mus musculus
aagaacctgctagaccacctg

SEQ ID NO: 2 - DNA, 20 bp, Mus musculus
tgcttcctcagtctgctcac

SEQ ID NO: 3 - DNA, 18 bp, Mus musculus
gcagcagcccagtacctc

SEQ ID NO: 4 - DNA, 18 bp, Mus musculus
gtcatcaggcacgaagca

SEQ ID NO: 5 - DNA, 22 bp, Mus musculus
cctcacatcttctccatctctg

SEQ ID NO: 6 - DNA, 22 bp, Mus musculus
ttggatgaccctcttagtgttg

SEQ ID NO: 7 - DNA, 25 bp, Mus musculus
tgacagtgatgagaatgacctgttc

SEQ ID NO: 8 - DNA, 20 bp, Mus musculus
ttggaagcagcccttcatct

SEQ ID NO: 9 - DNA, 25 bp, Mus musculus
gctaccaaactggatataatcagga

SEQ ID NO: 10 - DNA, 24 bp, Mus musculus
ccaggtagctatggtactccagaa

SEQ ID NO: 11 - DNA, 14 bp, Mus musculus
cggagtccggcagg

SEQ ID NO: 12 - DNA, 21 bp, Mus musculus
gctgggtagagaatggatgaa

SEQ ID NO: 13 - DNA, 19 bp, Mus musculus
gctccaagcagatgcagca

SEQ ID NO: 14 - DNA, 18 bp, Mus musculus
ccggatgtgaggcagcag

SEQ ID NO: 15 - protein, 13 aa, Artificial Sequence
  Artificially synthesized cyclic peptide
  MISC_FEATURE (1)..(13): The peptide is cyclized by a bond between amino acid positions 1 and 13
  MISC_FEATURE (9)..(9): The proline at amino acid position 9 is D-proline
  MISC_FEATURE (11)..(11): The amino acid at position 11 is a butylated norleucine
ASIPPQKYPPXET